(19) United States
(12) Patent Application Publication
(10) Pub. No.: US 2023/0405031 A1
(43) Pub. Date: Dec. 21, 2023

(54) METHODS AND COMPOSITIONS RELATED TO RECEPTOR-MEDIATED GLUCOSE SENSING

(71) Applicant: Ohio State Innovation Foundation, Columbus, OH (US)

(72) Inventors: Georgios KYRIAZIS, Columbus, OH (US); Joan SERRANO, Columbus, OH (US)

(21) Appl. No.: 18/037,929

(22) PCT Filed: Dec. 3, 2021
(86) PCT No.: PCT/US2021/061762

§ 371 Date: May 19, 2023

Related U.S. Application Data
(60) Provisional application No. 63/120,942, filed on Dec. 3, 2020.

(51) Int. Cl. A61K 31/7004; A61K 31/7105

(52) U.S. Cl. CPC A61K 31/7004; A61K 31/7105

(57) ABSTRACT

Disclosed herein is a method of modulating cell metabolism in skeletal muscle in a subject in need thereof, the method comprising: identifying a subject in need of modulation of cell metabolism in skeletal muscle; and administering to the subject a modulator of skeletal muscle in T1R2. Also disclosed herein is a method of identifying a modulator of T1R2 in skeletal muscle, the method comprising providing muscle-related cells, exposing the cells to a potential modulator of T1R2, and determining modulation, thereby identifying a modulator of T1R2. Further disclosed are compositions identified by this method.

### CROSS REFERENCE TO RELATED APPLICATIONS

This application claims the benefit of priority to U.S. Provisional Application No. 63/120,942, filed Dec. 3, 2020, which is incorporated herein by reference in its entirety.

### GOVERNMENT SUPPORT CLAUSE

This invention was made with government support under Grant No. R01 AR078264-01, awarded by the National Institutes of Health (NIH). The government has certain rights in the invention.

### SUMMARY

Disclosed herein is a method of modulating cell metabolism in skeletal muscle in a subject in need thereof, the method comprising: identifying a subject in need of modulation of cell metabolism in skeletal muscle; and administering to the subject a modulator of skeletal muscle in T1R2.

Also disclosed herein is a method of identifying a modulator of T1R2 in skeletal muscle, 20 the method comprising providing muscle-related cells, exposing the cells to a potential modulator of T1R2, and determining modulation, thereby identifying a modulator of T1R2. Further disclosed are compositions identified by this method.

### DESCRIPTION OF DRAWINGS

FIGS. 1A-1G show STR regulate glucose uptake in the skeletal muscle. (1A) Rate of systemic glucose appearance (Ra) and disappearance (Rd) and (1B) glucose uptake in skeletal muscle in conscious catheterized mice fasted for 5 h using isotopic enrichment (n=8-10/group). (1C) Liver glycogen content in fed and 5 hour (h) fasted mice (n=7/group). (1D) Gene expression in gastrocnemius muscle (HXK1/2, hexokinase; PFKAM, ATP-dependent 6-phosphofructokinase; PKM1, Pyruvate kinase; GYS1, Glycogen synthase; GSK3B, Glycogen synthase kinase-3 beta; PDK4, Pyruvate dehydrogenase 4; PHHA1, Pyruvate dehydrogenase E1 component alpha; CACP, Carnitine O-acetyltransferase) (n=8/group). (1E) and (1F) plasma insulin and glucagon, respectively in 5 h fasted mice (n=8-10/group). (1G) GLUT1 and GLUT4 protein levels in isolated membrane fractions (T-tubule) from gastrocnemius. Membrane protein and SERCA were used as loading controls (n=4/group). Student's T-test, p value shown.

FIGS. 2A-2F show STRs regulate skeletal muscle bioenergetics. Targeted quantitative metabolite profile of 5-hour fasted gastrocnemius (n=9-11/group). (2A) Partial Least Squares Discriminant Analysis (PLSDA), (2B) KEGG metabolic pathway analysis. Statistically significant and impactful pathway are shown in red dots. (2C) Significant metabolite differences in Pareto-scaled analysis using MetaboAnalyst 4.0. Floating bars show min to max values. T-test with FDR correction, p values shown at the bottom. (2D, 2E) Uric acid, NADP and NADPH concentrations. (2F) Schematic of pathways relevant to metabolite profiles (G6P, glucose-6-phosphate; HK, hexokinase; PARG, Poly(ADP)ribose glycohydrolase; NAM, nicotinamide; NMN, nicotinamide mononucleotide).

FIGS. 3A-3B show STRs regulate PARP1 activity in skeletal muscle. (3A) Gene expression in gastrocnemius muscle of ad lib fed mice (NADK2, NAD kinase 2; PARP1/2, Poly [ADP-ribose] polymerase 1,2; SIRT1/2, NAD-dependent protein deacetylase sirtuin-1/2; CD38, ADP-ribosyl cyclase; NAMPT, Nicotinamide phosphoribosyltransferase; NMNAT1/2/3, Nicotinamide mononucleotide adenylyltransferase 1/2/3; NUTD5, Adp-ribose pyrophosphatase; NADSYN1, Glutamine-dependent NAD(+) synthetase; G6PD1, Glucose-6-phosphate 1-dehydrogenase; PRPS1, ribose phosphate pyrophosphokinase; PPAT, phosphoribosyl pyrophosphate amidotransferase; PPP, pentose phosphate pathways) (n=8/group). (3B) Immunoblot of auto-poly (ADP) ribosylation (polyADPr), PARP1, NAMPT and tubulin as loading control. Quantitation of PARP1 activity (polyADPr/PARP1 adjusted for tubulin) (n=6/group). Student's T-test, p value.

FIGS. 4A-4H show STRs regulate mitochondrial function and exercise capacity. (4A) Oxygen flux in muscle fibers in response to malate+pyruvate and ADP. Addition of antimycin A suppressed oxygen flux (not shown) (n=8-12/group) Two-way ANOVA, post hoc, p value. (4B) Oxygen consumption rate (OCR) in differentiated myoblasts. Oligomycin (Comp V inhibition), FCCP (Carbonyl cyanide-4 (trifluoromethoxy) phenylhydrazone; uncoupler), rotenone (Comp I inhibition) (n=5/group). (4C) Quantitative analysis of lactate, ATP and ADP in gastrocnemius following 5-hours fasting (n=9-11/group). (4D) Succinate dehydrogenase staining in muscle sections. Arrows show SDH staining in gastrocnemius n=6/group). (4E) Immunoblotting of mitochondrial complex proteins with quantitation (n=6/group). (4F) Exercise endurance in mice at 10°,% grade and 20 m/min (n=8/group). (4G) Oxygen consumption and (4H) respiratory exchange ratio (RER) under steady state conditions (plateaued values for at least 10 min) before exhaustion in mice subjected to a submaximal exercise bout as in (4F). Student's t-test, p value for (4B-4H).

FIGS. 5A-5C show STRs expression in skeletal muscle. STR (T1r2 and T1r3) expression in (5A) quadriceps (Quad), tibialis anterior (TA), extensor digitorum longus (EDL), gastrocnemius (Gastroc), soleus, plantaris muscles (n=6/group), (5B) primary cultures of differentiating myoblasts (n=3/group), and (5C) primary cultures of differentiated human myotubes (n=3).

FIGS. 6A-6D show phenotype of muscle-specific deletion of T1r2 gene. (6A) T1r2 exon2 floxed mice were crossed with mice expressing Cre recombinase under the myogenin promoter. Recombination occurred only in skeletal muscles producing the predicted 800 bp band and causing ablation of T1r2 expression (not shown). (6B) Plasma glucose in response to 5 h fasting (n=10/group). (6C) Exercise endurance in mice (n=6-8/group). Student's t-test, p value (6D) Immunoblot of auto-poly ADP ribosylation (polyADPr) in gastrocnemius.

FIG. 7 shows schematic of proposed STR-mediated signaling pathways. STRs activate PLC-mediated cascade that leads to ERK-dependent regulation of PARP activity and NAD levels. In turn, this alters the function of SIRT1 and SIRT2 causing adaptations in mitochondrial function and glucose utilization towards nucleotide biosynthesis, respectively.

FIGS. 8A-8E show STR signaling involves ERK2 and SIRT1 activation. (8A) ERK1/2 phosphorylation in gastrocnemius from fed mice. Quantitation includes both ERK1 and ERK2 bands (n=6/group). (8B-8C) ERK2 phosphorylation in response to bilateral intramuscular injection of either sucralose or 3-OMG (right gastroc) or vehicle (left gastroc) in anesthetized mice after an overnight fast (n=8/group). (8D) NAD levels in C2C12 cells culture in high (black dots) or low (blue dots) glucose for 54 h. In select cells, equal molarity (20 mM) of glucose (Glu) or 3-OMG was added during the last 6 h (ANOVA). (8E) PGC1a acetylation in gastrocnemius from 5 hours fasted mice using immunoprecipitation (IP) with anti-PGC1a (Millipore ST1202) and then immunoblotting (IB) with anti-PGC1a and anti-acetyl-lysine (CST #9441). IB of PGC1a is shown in input. Student's T-test, p value.

FIGS. 9A-9F show ablation of STRs increases myofiber size. (9A) Body composition (fat and lean mass) using EchoMRI analyzer (n=14-18/group). (9B), (9C) and (9E) myofiber size distribution using cross sectional area (CSA). Inset shows the geometric mean of fiber CSA (n=6/group). (9D) myofiber type distribution using CSA (n=6/group). (9F) IP.GTT in 5 hours fasted mice using Ig/kg (n=16-20/group). For (9F) Two-way ANOVA, p value for time x genotype interaction. *post hoc p<0.05. For (9A-9D), Student's t-test, p value.

FIGS. 10A-10C show ablation of STR signaling potentiates protein synthesis during refeeding. (10A) Phosphorylation of 4E-BP1 and rpS6 adjusted for total protein, respectively and tubulin using immunoblotting, arbitrary units (AU), or (10B) Expression of STR (T1r2 and T1r3) in gastrocnemius muscle of mice fasted (F) o/n or following refeeding (RF) for 2 hours. Relative quantitation shown. Two-way ANOVA; for (10A) post hoc p values shown (n=3-4/group), for (10B) ANOVA p value (n=5-6/group). (1° C.) Puromycin incorporation using immunoblotting as a measurement of protein synthesis in vivo, adjusted for total ponceau. Mice fasted for 5 hours were i.p. injected with puromycin and 30 minutes later the gastrocnemius muscle was harvested. Student's t-test, p value.

FIGS. 11A-11B show effects of STR signaling in response to compensatory hypertrophy. (11A) Dry weight of plantaris (PLA) muscle 14-days following synergist ablation surgery or sham. ANOVA surgery main effect, p value (n=4-6/group) (11B) Phosphorylation of 4E-BP1 and rpS6 adjusted for total protein respectively using immunoblotting following ablation or sham (sh). Arbitrary units (AU) shown. Student's T-test, p-value (n=5/group).

FIGS. 12A-12C show mRNA using RiboTag technology, postnatal gene expression, and mT/mGT1r2 reporter mouse validation. (12A) Skeletal muscle-specific mRNA enrichment (Log 10) relative to whole-muscle input control (set at 1) in actively transcribed mRNA pulled down from RiboTag-Myog mice. Expression enrichment of T1r2, the muscle-specific marker, Myh2, and the macrophage-specific marker, CD206 (negative control) (n=3/group). (12B) Relative STR expression in the gastrocnemius of WT postnatal mice (Adult expression set at 1 (n=3/group; Two-ANOVA, time x genotype interaction, p<0.0001). (12C) T1r2+ cells (green) in taste buds (circled area) and cross sections of soleus muscle. Prior to T1r2-Cre recombination, cell membrane-localized tdTomato (mT) fluorescence expression is widespread in cells/tissues (red). Cre recombinase expressing cells (and future cell lineages) have cell membrane-localized EGFP (mG) fluorescence expression replacing the red fluorescence. Blue arrow: T1R2+ cells (green). Yellow arrow: sagittal view of T1R2+(green) skeletal muscle fibers of the tongue. Laser intensity and contrast adjustments are equal across photos.

FIG. 13 shows T1R2 wild-type versus T1R2 knockout mouse models. Both models show fed versus fasted mice, and it can be seen that the T1R2 knockout model shows spontaneous improvements in muscle plasticity when compared to the wild type T1R2 mice.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibitors,” “activators,” and “modulators” of chemosensory receptor, e.g., T1R2, are used interchangeably to refer to inhibitory, activating, or modulating molecules identified using in vitro and in vivo assays for chemosensory signal transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate taste transduction, e.g., antagonists. Activators are compounds that, e.g., bind to, stimulate, increase, open, activate, facilitate, enhance activation, sensitize, or up regulate chemosensory signal transduction, e.g., agonists. Modifiers include compounds that, e.g., alter, directly or indirectly, the activity of a receptor or the interaction of a receptor with its ligands, e.g., receptor ligands and optionally bind to or interact with activators or inhibitors. Modulators include genetically modified versions of chemosensory receptors, e.g., T1R2, e.g., with altered activity, as well as naturally occurring and synthetic ligands, antagonists, agonists, small chemical molecules and the like.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, to “alleviate” a disease means to reduce the frequency or severity of at least one sign or symptom of a disease or disorder.

An “effective amount” as used herein, means an amount which provides a therapeutic or prophylactic benefit.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

As used herein, the term “cell” is herein used in its broadest sense in the art, referring to a structural unit of a tissue present in a multicellular organism, which is capable of self-replicating, has genetic information and a mechanism for expressing it, and is surrounded by a membrane structure that isolates the living body from the outside. Cells used herein may be either naturally-occurring cells or artificially modified cells (e.g., fusion cells, genetically modified cells, etc.), as long as the cell has a chemical receptor or is capable of having such a nucleic acid molecule introduced therein. Examples of cell sources include, but are not limited to, a single-cell culture; the embryo, blood, or a body tissue of a normally-grown transgenic animal, a mixture of cells derived from normally-grown cell lines, and the like. Specifically disclosed herein are muscle cells, such as a myocyte or myoblast.

As used herein, the term “tissue” refers to an aggregate of cells having substantially the same function and/or form in a multi-cellular organism. “Tissue” is typically an aggregate of cells of the same origin, but may be an aggregate of cells of different origins as long as the cells have the same function and/or form. Typically, a tissue constitutes a part of an organ. Animal tissues are separated into epithelial tissue, connective tissue, muscular tissue, nervous tissue, and the like, on a morphological, functional, or developmental basis. Specifically contemplated herein is muscle tissue.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are subsequently chemically synthesized.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

General Description Skeletal muscle function and mass readily adapts to environmental stimuli by integrating mechanical, hormonal, neuronal and metabolic pathways. This remarkable plasticity involves the activation of intracellular sensors aiming to satisfy energy demands and balance anabolic and catabolic pathways. Nicotinamide adenine dinucleotide (NAD*) is an endogenous metabolite involved in these processes by participating in redox reactions and by serving as a substrate for poly(ADP-ribose) polymerases (PARPs) and NAD-dependent deacetylases (sirtuins; SIRT1,3,6) (1). Depletion of cellular NAD⁺ is linked to insulin resistance, diabetes (2), skeletal muscle dysfunction and mass loss (3), while strategies that restore or increase its levels can reverse this pathogenesis (4). Because PARPs and SIRTs compete for NAD⁺, genetic or pharmacological inhibition of PARP1 is one strategy to boost NAD⁺, leading to SIRT1 activation and its downstream beneficial outcomes (5, 6). Thus, identifying metabolic pathways that regulate PARP1 activity can help the development of innovative therapies for the prevention or treatment of muscle degeneration and metabolic dysfunction. Disclosed herein is the finding that direct sensing of circulating glucose by sweet taste receptors (STRs) regulates PARP1 activity to control the adaptive potential of skeletal muscle.

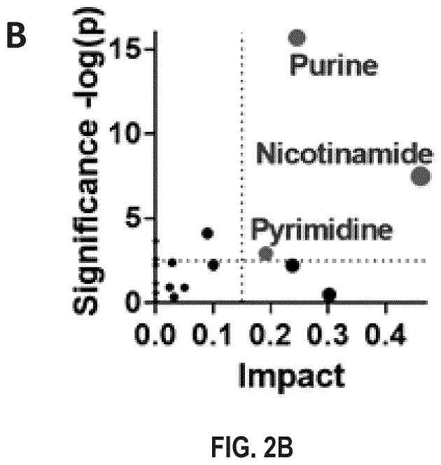

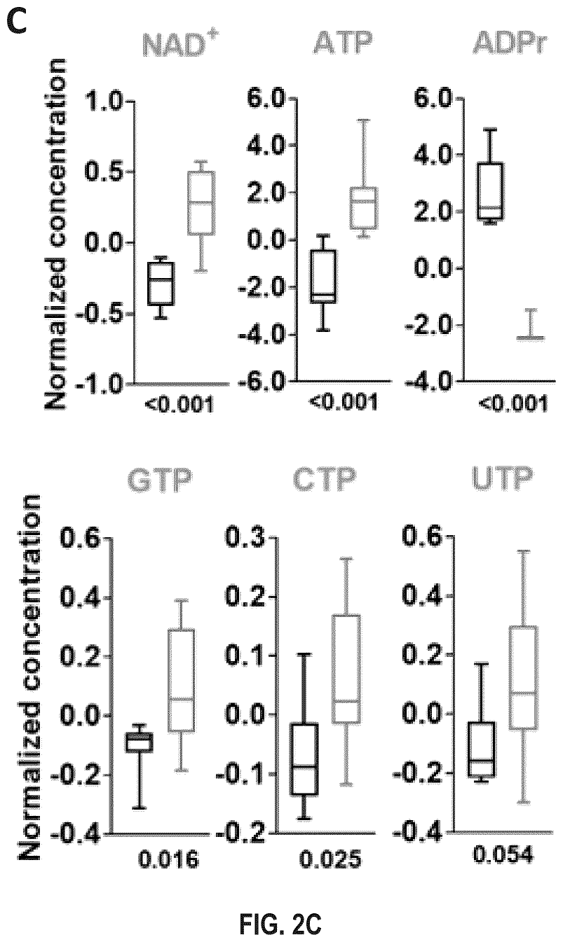

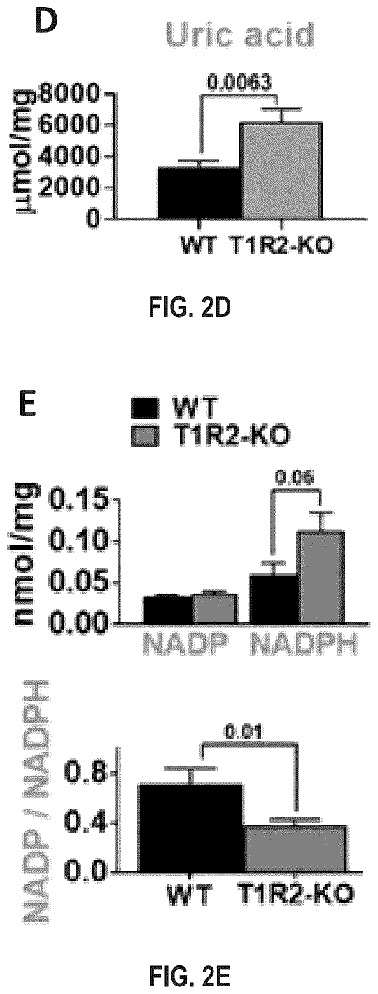

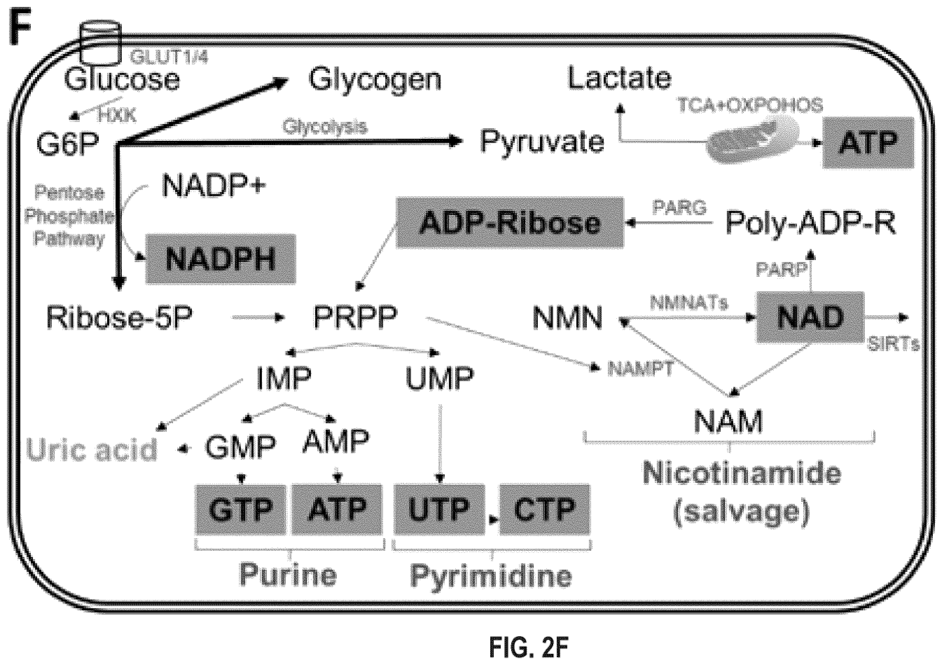

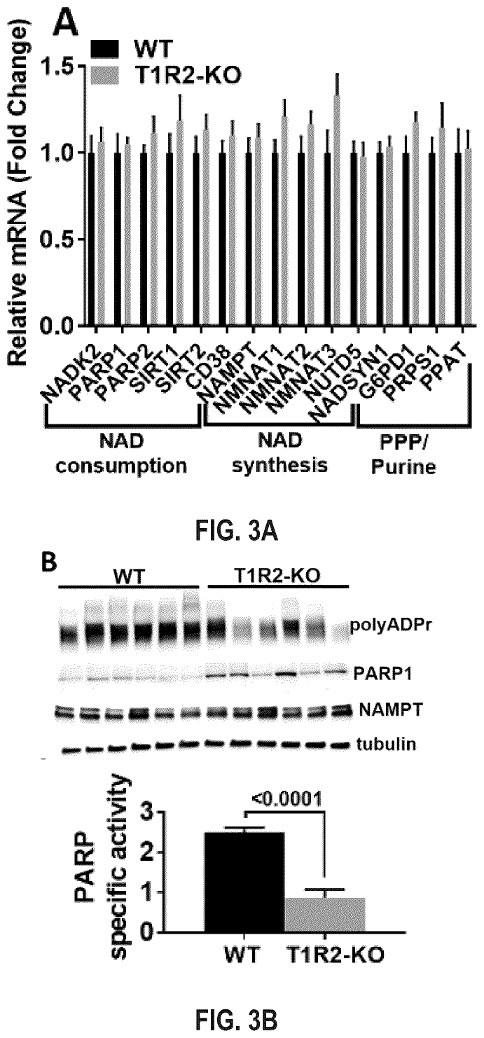

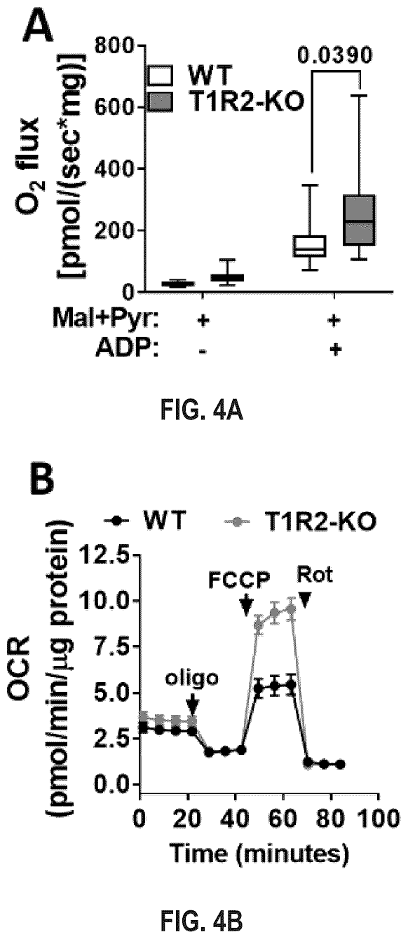

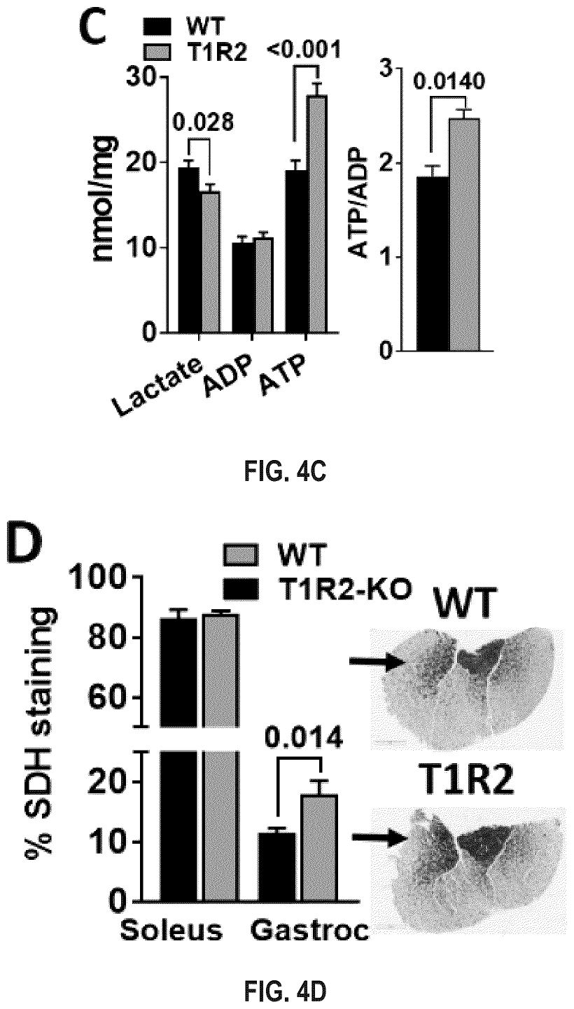

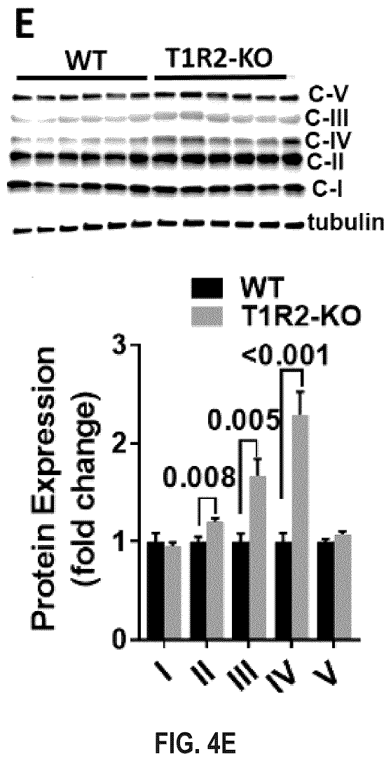

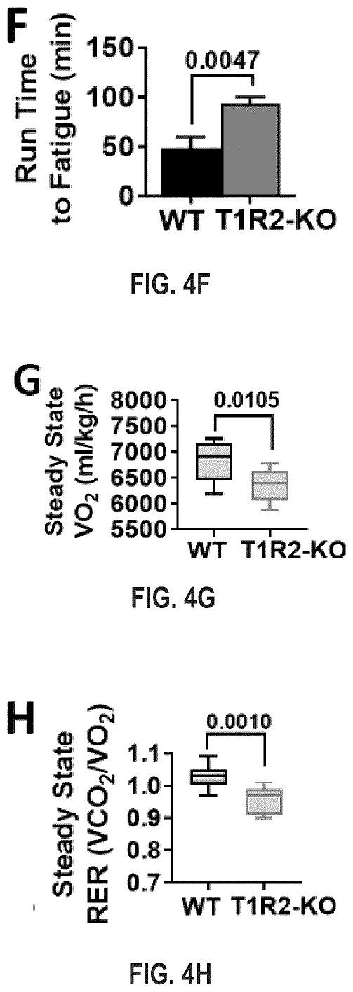

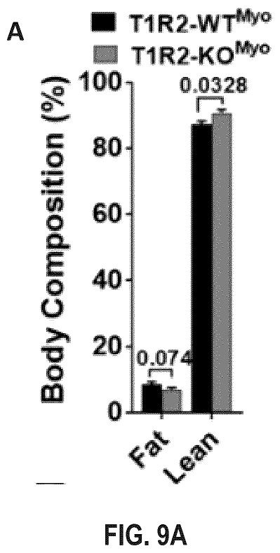

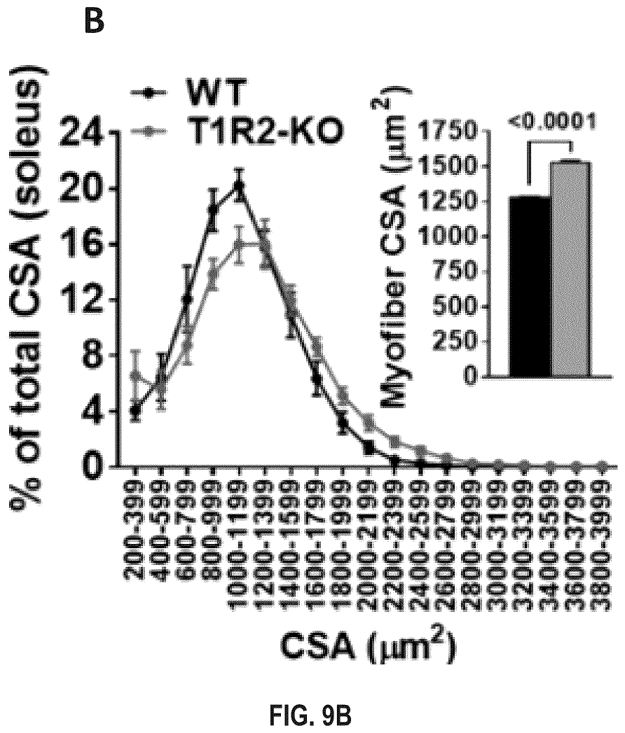

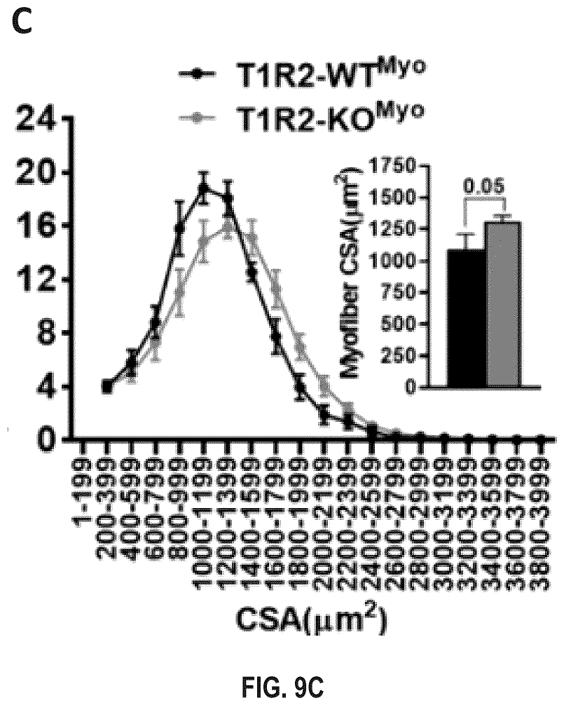

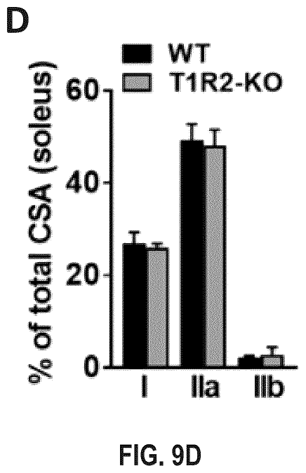

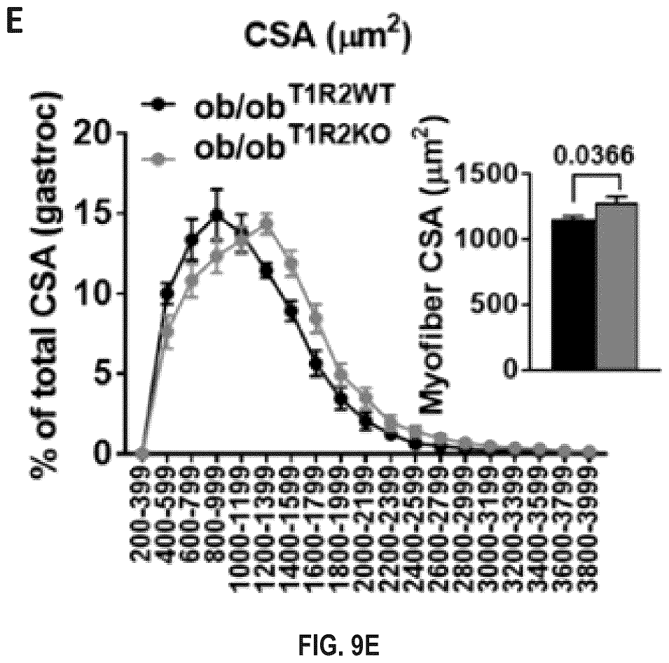

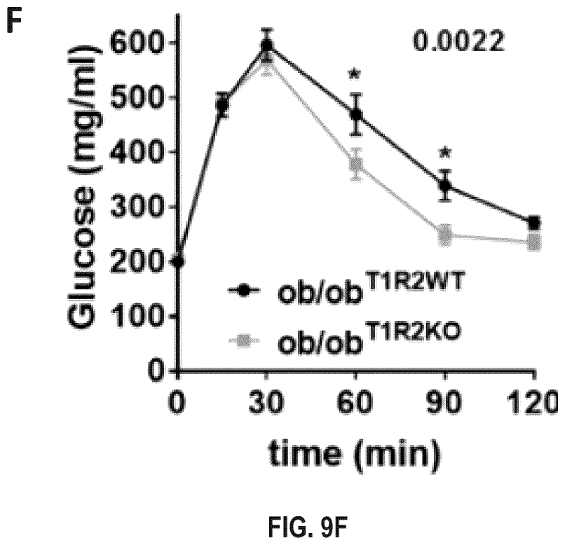

STRs are G protein-coupled receptors (GPCRs) formed by the obligate heterodimerization of T1R2/T1R3 proteins. Beyond the tongue, STRs can sense ingested or circulating nutrients to exert pleiotropic effects (7, 8). For instance, it was previously demonstrated that STR-mediated glucose or fructose sensing on endocrine cells regulates insulin (9, 10) and incretin (11) secretion. Now, evidence is provided showing that the role of STRs is not limited to endocrine regulation, but it is also instrumental in the control of cell metabolism through STR-mediated glucose sensing in skeletal muscle. Whole body (T1R2-KO) or skeletal muscle-specific (T1R2-KO^{Myog}) deletion of T1r2 gene of STRs enhances mitochondrial function, oxidative capacity, exercise tolerance, and induces mild increases in myofiber size (FIGS. 4 and 9). These improvements are linked to attenuated PARP1 activity, increased ATP and NAD⁺ pool, and a shift in glucose utilization towards nucleotide biosynthesis (FIGS. 2 and 3).

Consequently, genetic ablation of STRs in ob/ob mice (genetic obesity) partially prevented muscle mass loss and improved glucose tolerance (FIG. 9). T1R2-KO mice are protected from metabolic derangements associated with diet-induced obesity, including maintenance of lean mass (12). It is disclosed herein that the T1R2 receptor is a constitutive sensor of glucose availability to adjust intracellular pathways that control the metabolic basis of skeletal muscle plasticity.

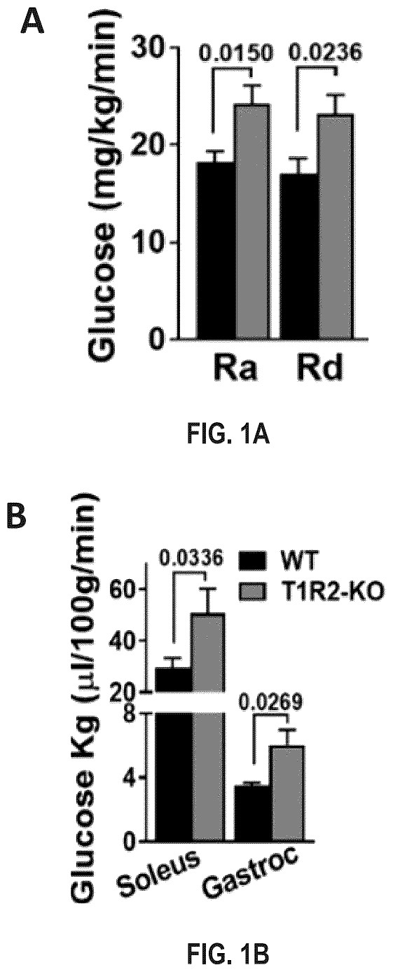

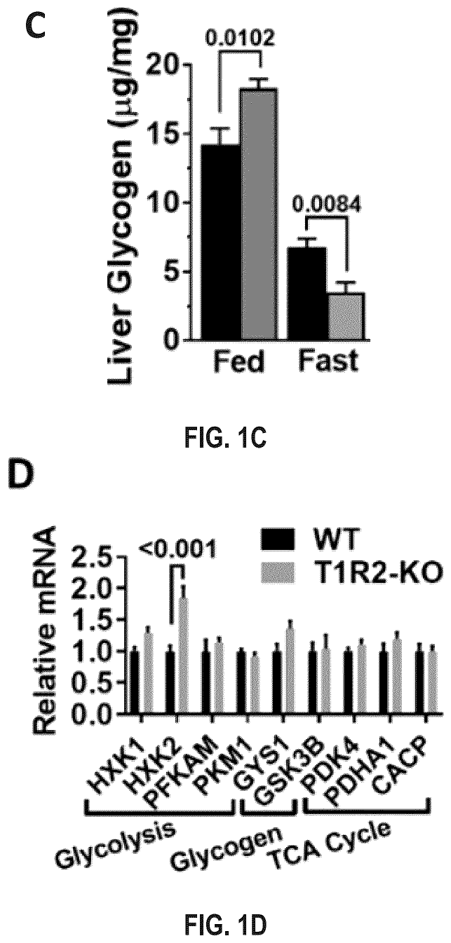

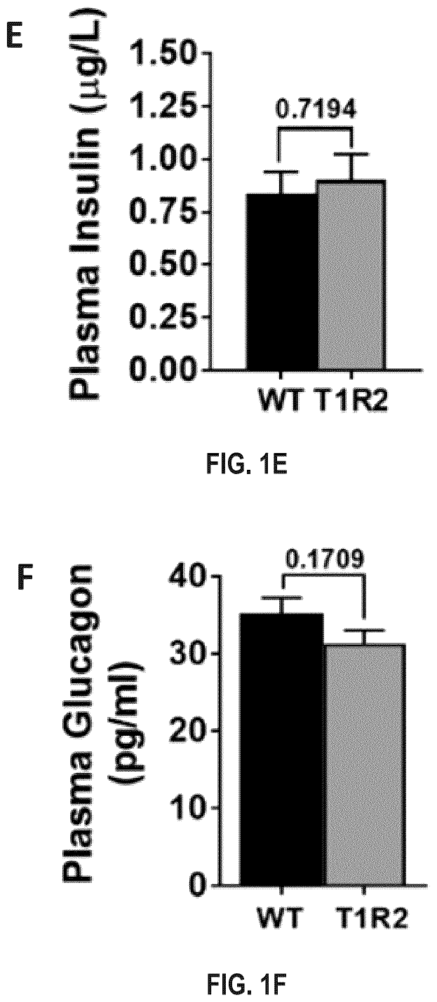

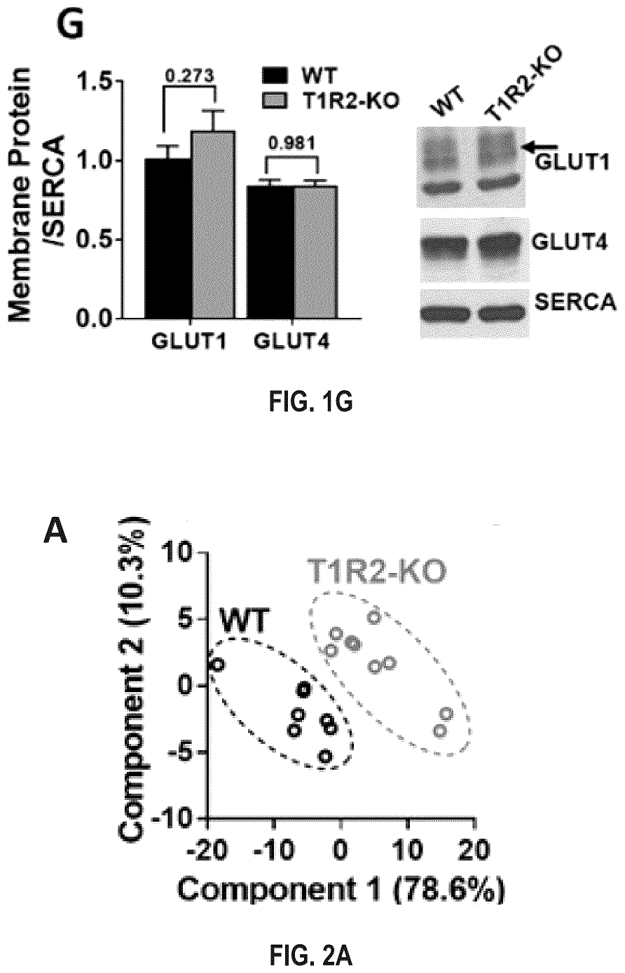

STRs are constitutive glucose sensors that signal to regulate NAD⁺ metabolism and the fate of glucose utilization in skeletal muscle. STR-mediated glucose sensing contributes to the regulation of anabolic responses to adaptive stimuli. Adult T1R2-KO and T1R2-KO^{Myog} muscles have increased pool of anabolic intermediates, enhanced protein synthesis, and mild increases in myofiber size. Disclosed herein is the finding that skeletal muscle can directly sense systemic glucose through membrane GPCRs. Like other cell types, skeletal muscle deploys a network of glucose sensing mechanisms aiming to couple energy availability to cell homeostasis. It was previously thought that these mechanisms are entirely dependent on glucose uptake and metabolism to generate numerous glucose-derived metabolites that are detected by a variety of cellular sensors (13-18). Now, it is shown that sweet taste receptors (STRs) on skeletal muscle are direct sensors of extracellular glucose that constitutively couples nutrient availability to the regulation of skeletal muscle mass and function (plasticity) (FIGS. 1, 4, and 9).

NAD⁺ bioavailability is central to the maintenance of metabolic health, in part, due to the activation of the NAD⁺-SIRT axis (19). Thus, approaches that either enhance NAD⁺ synthesis (i.e. NAD⁺ precursor supplementation (4, 20), exercise (21, 22), caloric restriction (18, 23, 24)) or inhibit its consumption (i.e. PARPs (5, 6, 25), CD38 (26, 27)) can lead to beneficial metabolic outcomes, such as improvements in mitochondrial function, metabolic flexibility, insulin sensitivity and muscle mass. Particularly, genetic or pharmacological inhibition of PARP1, a major NAD⁺ consumer, improves muscle fitness (5, 6). Nevertheless, the physiological pathways that link energy metabolism to PARP1 activity for the coordination of NAD⁺ bioavailability were not previously described. Disclosed herein is the finding that STR signaling targets PARP1 activity to adjust NAD⁺ levels and SIRT activation (FIGS. 2, 3, and 4). This mechanism links, for the first time, extracellular glucose availability to metabolic and functional adaptations in skeletal muscle.

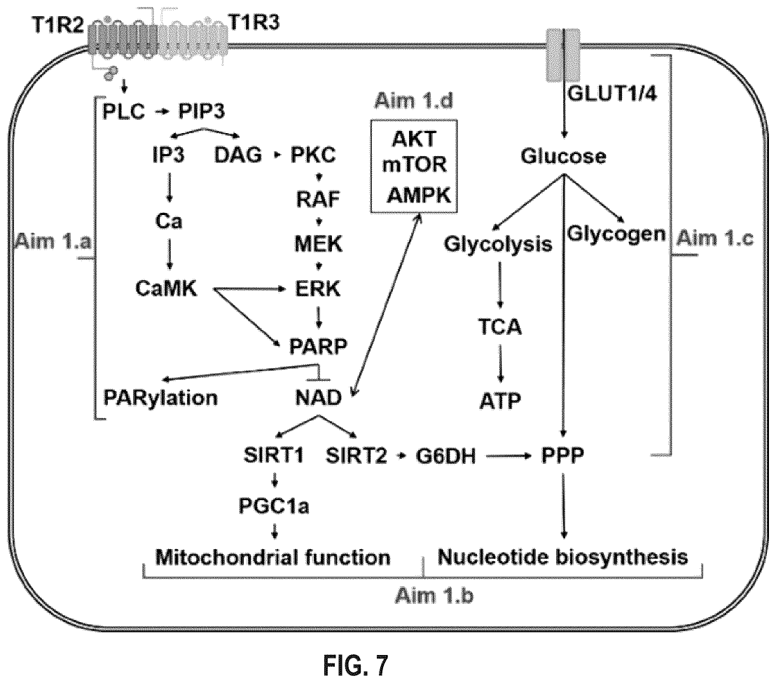

Upon its transport and phosphorylation in the skeletal muscle, glucose can be a) oxidized to meet energy demands, b) stored as glycogen for future use, or c) converted to other sugars to meet the needs of biosynthetic/anabolic pathways (28) (FIG. 7). This latter fate of glucose utilization can be crucial during growth since glucose shunt through the pentose phosphate pathway (PPP) provides the ribose moiety (5-phosphorybosyl-1-pyrophosphate; PRPP) that is indispensable for purine and pyrimidine biosynthesis (29-31) (FIG. 2F). Excess glucose disposal in the skeletal muscle bolsters its oxidation and/or storage (32, 33), but high glucose influx per se is not sufficient to induce nucleic acid biosynthesis (31). Disclosed herein is that, in skeletal muscle, glucose flux and utilization towards biosynthetic pathways is regulated in part by STRs and depends on the systemic availability of glucose (FIGS. 1 and 2). This novel mechanism constantly informs myocytes about peripheral energy status to modulate, along with other pathways, the metabolic basis for muscle maintenance.

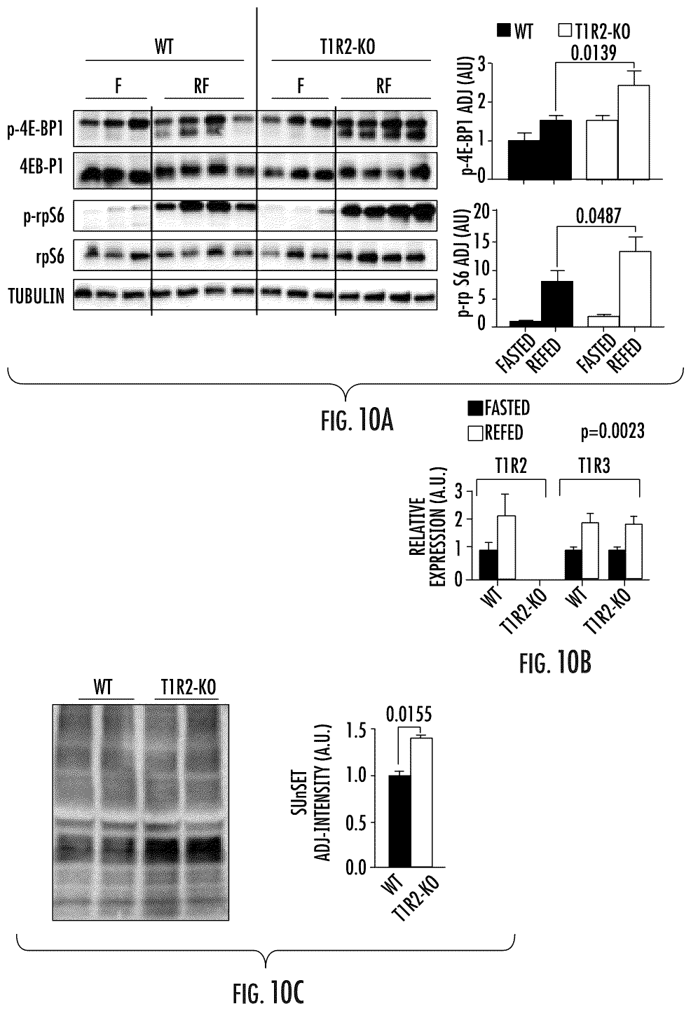

Skeletal muscle is a major site for the development of metabolic dysfunction during type 2 diabetes (T2D) and obesity (reviewed in (34, 35). Notably, these conditions are often accompanied by accelerated muscle loss (36, 37) despite the presence of nutrient abundance. Among other things, this suggests the uncoupling of nutrient sensing mechanisms with the molecular pathways that control muscle plasticity. Thus, it is critical to identify and target molecular networks that preserve or restore this axis during the development of metabolic or age-related dysfunction. Towards this end, it is shown that T1R2-KO or T1R2-KO^{Myog} muscles have mild increases in myofiber size (15-20%) and demonstrate enhanced rates of protein synthesis (FIGS. 9 and 10). This phenotype is consistent with a report showing that T1R2-KO mice resisted lean mass losses associated with chronic high-fat diet (HFD) feeding (12). In addition, genetic deletion of STRs in ob/ob mice—a model of obesity and metabolic dysfunction (38, 39)—also partially prevented muscle mass loss and improved glucose tolerance (FIG. 9). It appears that the chronic and sustained hyperglycemia in T2D and obesity can constitutively hyper-activate STR signaling in the skeletal muscle contributing to muscle mass loss. Taken together, these findings show that STR-mediated glucose sensing is implicated in the development of muscle mass dysfunction and deterioration.

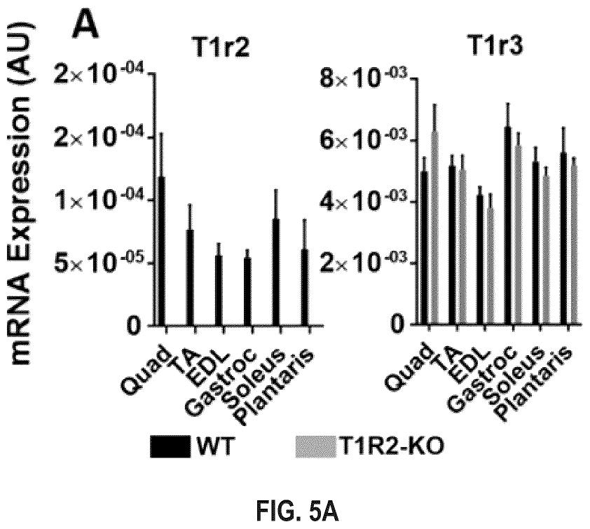

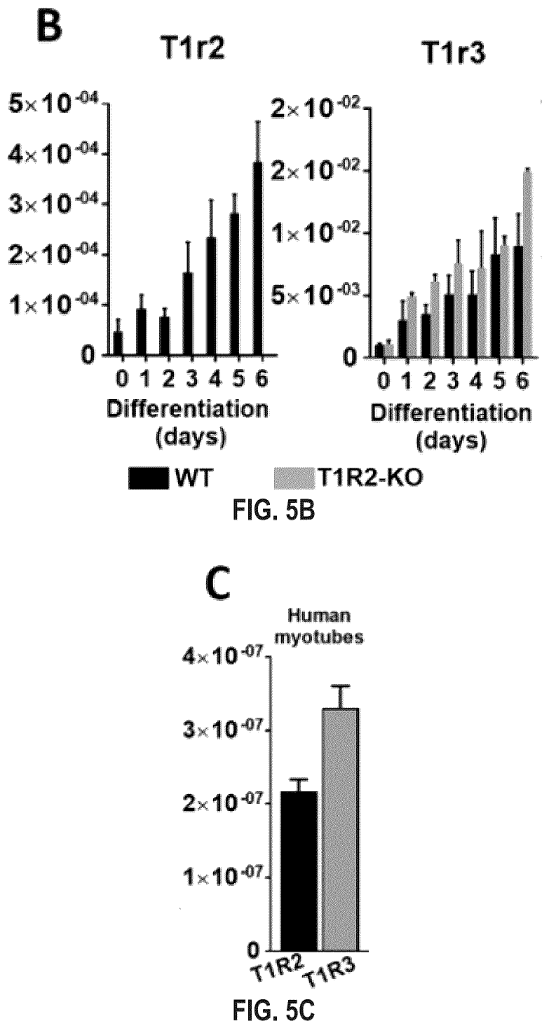

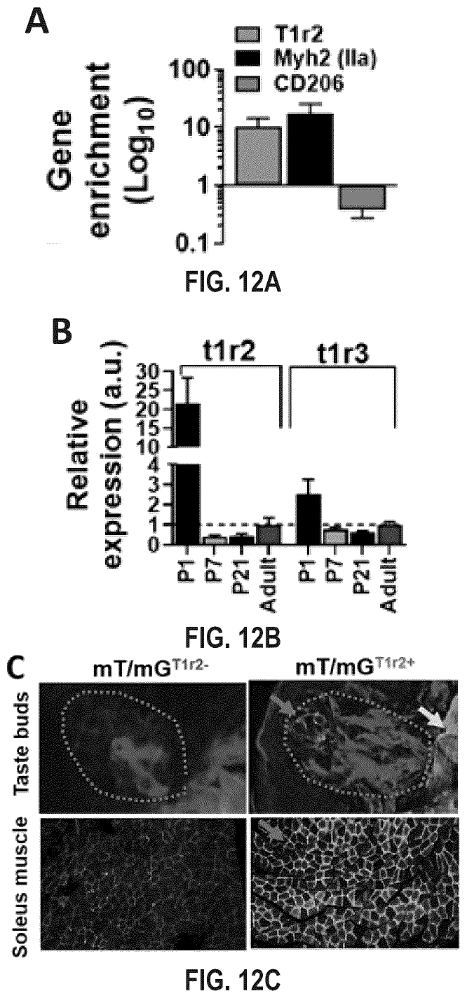

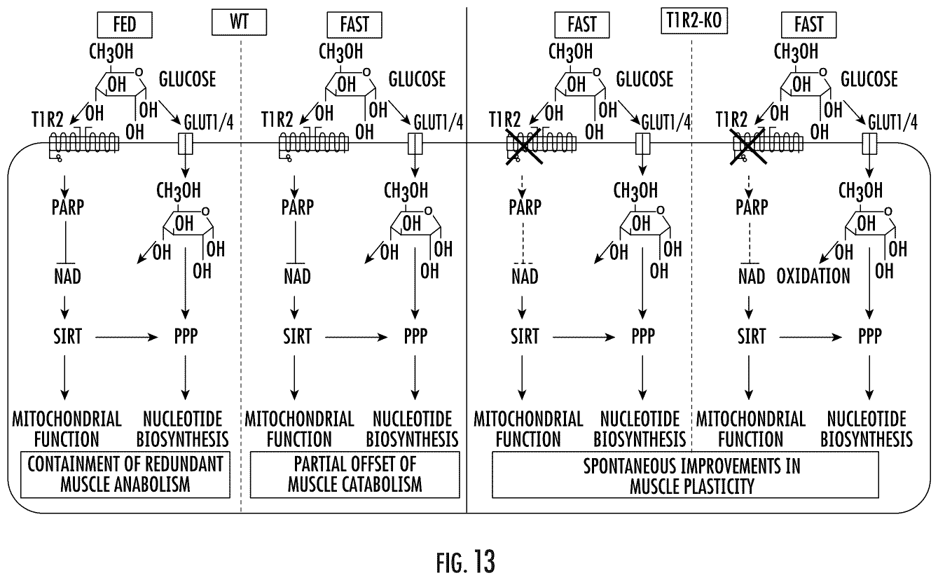

Disclosed herein are STRs as a novel therapeutic target for muscle dysfunction. It is known that transient inhibition of PARP1 can improve muscle fitness and metabolism (5, 6). It is now shown that ablation of STRs also inhibits PARP1 to recapitulate identical effects (FIGS. 3, 4, and 9), showing that both STRs and/or PARP1 can be promising targets for muscle dysfunction. However, PARP1 is regulated by numerous signals to control various processes, such as DNA repair and transcription (40). So, chronic pharmacological inhibition of PARP1 may indiscriminately interfere with these essential functions, counteracting the desirable effects on muscle metabolism. Alternatively, inhibition of STRs can specifically attenuate PARP1 activity linked to metabolic control, without disrupting other signals that regulate PARP1 or essential functions of PARP1. This therapeutic approach is also appealing because GPCRs (i.e. STRs) are excellent targets for pharmacological interventions (41). Beyond the tongue, STRs are expressed in the pancreas (9, 10, 42), intestine (11, 43-46), adipose tissue (47, 48) and, as now shown, skeletal muscle (FIGS. 5 and 12).

In summary, when peripheral glucose is increased, such as in the fed state, STR signaling is fully activated to suppress NAD⁺ levels and mechanisms that promote glucose utilization towards nucleic acid biosynthesis. This bioenergetic checkpoint ensures that the increased influx of glucose during the fed state is not unnecessarily consumed in anabolic pathways causing redundant muscle growth and/or futile energy cycles. In contrast, when peripheral glucose is decreased, such as during fasting, STR signaling is progressively attenuated to boost NAD⁺ levels and sirtuin activation, and to relatively promote glucose utilization towards nucleotide biosynthesis. This can be a physiological adaptation to partially offset the global muscle catabolism that set in during energy shortage. However, muscles with genetic ablation of STRs (T1R2-KO) are insensitive to peripheral glucose fluctuations (fed or fasted). Consequently, during feeding, STR-deficient muscles cannot sense the high circulating glucose to activate the “break” in NAD⁺-dependent pathways and glucose shunt to biosynthesis. Over time, constitutive activation of these downstream pathways in T1R2-KO mice causes beneficial adaptations in muscle function and mass (plasticity). Experimental evidence supporting this model is shown in Examples 1 and 2.

Methods and Compositions for Modulating Cell Metabolism in Skeletal Muscle

Disclosed herein is a method of modulating cell metabolism in skeletal muscle in a subject in need thereof, the method comprising: identifying a subject in need of modulation of cell metabolism in skeletal muscle; and administering to the subject a modulator of skeletal muscle in T1R2. Modulation of the T1R2 can comprise inhibition or downregulation of T1R2.

Such inhibition or downregulation can comprise abrogation of glucose sensing by T1R2.

Modulation can also cause upregulation, or an increase, in T1R2 activity.

Importantly, specifically regulating T1R2 activity in muscle cells has never been reported. Kokabu et al. (Kokabu S, Lowery J W, Toyono T, Sato T, Yoda T. On the Emerging Role of the Taste Receptor Type 1 (T1R) Family of Nutrient-Sensors in the Musculoskeletal System. Molecules. 2017; 22(3):469) disclose T1R3 being expressed in skeletal muscle cell lines, but a function relevant to T1R2/T1R3 was not mentioned, revealed or inferred. In addition, there is no reference regarding T1R2 gene or its function. Notably, T1R3 can also heterodimerize with T1R1 to mediate amino acid sensing (i.e. umami taste). Consequently, manipulation of T1R3 cannot provide specificity since it is a common receptor subunit for both STRs (i.e. T1R2/T1R3) and amino acid receptors (i.e. T1R1/T1R3). Thus, manipulation of T1R2 is specific to STR signaling (i.e. T1R2/T1R3) without interfering with amino acid signaling (i.e. T1R1/T1R3). Most importantly, these nutrient receptors are also activated by different non-overlapping ligands. Glucose and other sugars are ligands for T1R2/T1R3, while amino acids are ligands for T1R1/T1R3.

When downregulation, or inhibition, of T1R2 by a modulator occurs, this can cause a 5, 10, 15, 20, 25, 30, 35, 40, 45, 50, 55, 60, 65, 70, 75, 80, 85, 90, 95, or 100% inhibition when compared to a control, or any amount below or between these amounts. The control to which it is compared is T1R2 which has not been exposed to an inhibitor.

When upregulation, or activation, of T1R2 by a modulator occurs, this can cause a 5, 10, 15, 20, 25, 30, 35, 40, 45, 50, 55, 60, 65, 70, 75, 80, 85, 90, 95, or 100% or more increase when compared to a control, or any amount below or between these amounts. The control to which it is compared is T1R2 which has not been exposed to an activator.

By “glucose sensing” is meant sensing any substance which can elucidate a response from T1R2. In other words, T1R2 is able to “sense” a substance and triggers a change in signal transduction, for example. Substances that can cause this response in T1R2 include, but are not limited to glucose, as well as any other natural or synthesized sweet flavor compound including non-caloric sweet flavor compounds, reduced caloric sweet flavor compounds, non-target caloric sweet flavor compounds, etc. Exemplary sweet flavor compounds include, without any limitation, cyclamic acid, mogroside, tagatose, maltose, galactose, mannose, sucrose, fructose, lactose, aspartame, neotame and other aspartame derivatives, saccharin, sucralose, acesulfame K, glucose, erythritol, D-tryptophan, glycine, mannitol, sorbitol, maltitol, lactitol, isomalt, hydroganeted glucose syrup (HGS), hydrogenated starch hydrolyzate (HSH), stevioside, rebaudioside A and other sweet Stevia-based glycosides, alitame, carrelame and other guanidine-based sweeteners, tagatose, xylitol, high fructose corn syrup, etc.

Modulation of cell metabolism can cause multiple effects in a cell or within a subject such as a human to which the modulator has been given. For example, modulation can comprise modulation of intracellular NAD levels. As discussed above, depletion of cellular NAD⁺ is linked to insulin resistance, diabetes, skeletal muscle dysfunction and mass loss, while strategies that restore or increase its levels can reverse this pathogenesis. Regulation of intracellular NAD⁺ levels are useful in treating or alleviating a symptom of various disorders in which aberrant (i.e., increase or decrease) mitochondrial function is involved. For example, regulation of intracellular NAD⁺ levels is useful in treating or alleviating a symptom of mitochondrial disorders which include diseases with inherited and/or acquired mitochondrial dysfunction, such as Charcot-Marie-Tooth disease, Type 2A2, Mitochondrial Encephalopathy Lactic Acidosis and Stroke (MELAS), Leigh Syndrome, Barth Syndrome, Leber's optic neuropathy, fatty acid oxidation disorders, inherited forms of deafness and blindness, metabolic abnormalities induced by exposure to toxic chemicals and/or drugs (e.g. cisplatin induced deafness, gentamycin induced deafness).

Also disclosed is modulation of poly ADP ribose polymerase (PARP) levels, such as PARP1. Because PARPs and SIRTs compete for NAD⁺, genetic or pharmacological inhibition of PARP1 can boost NAD⁺, leading to SIRT1 activation and its downstream beneficial outcomes. PARP1 constitutes one of the major NAD⁺ consumers in the cell. PARP-1 is activated upon binding to damaged or abnormal DNA, and catalyzes the formation of poly(ADP-ribose) polymers (PAR) onto different acceptor proteins, including PARP1 itself (auto-PARylation), using NAD⁺ as substrate. A reduction or ablation of PARP1 activity increases NAD⁺ levels and SIRT1 activity, which, in turn, promotes mitochondrial content and function, culminating in a solid protection against metabolic disease (U.S. Patent 2017/0182076A1, herein incorporated by reference in its entirety for its teaching concerning PARP1).

Modulating T1R2 in skeletal muscle of a subject (such as a human) can lead to beneficial effects in that subject. For example T1R2 inhibition can lead to increased oxidative capacity, increased exercise tolerance, and increased muscle fiber mass and/or size. This increase can be 10, 20, 30, 40, 50, 60, 70, 80, 90, or 100% or more, or any amount below, between or above these amounts. High oxidative capacity in muscle is important in maintaining muscle fuel source flexibility, and has been positively correlated with insulin sensitivity. Zampino et al. (Marta Zampino et al. Greater Skeletal Muscle Oxidative Capacity Is Associated With Higher Resting Metabolic Rate: Results From the Baltimore Longitudinal Study of Aging, The Journals of Gerontology: Series A, Volume 75, Issue 12, December 2020, Pages 2262-2268), incorporated by reference in its entirety for its teaching regarding mitochondrial dysfunction, discusses how mitochondrial dysfunction is considered a major hallmark of aging. Mitochondria provide the majority of energy for biological processes in the form of adenosine triphosphate (ATP), which can be dephosphorylated causing release of free energy. In skeletal muscle, mitochondrial density, O₂ consumption at peak exercise, and tricarboxylic acid cycle enzyme activity decline with aging. Such decline is associated with dysfunction of the electron transport chain and decreased ATP production, leading to energetic deficits. This age-related reduction of oxidative capacity in skeletal muscle, due in large part to mitochondrial dysfunction, is considered an important factor driving muscle aging and sarcopenia. By way of specific example, the subject may have been diagnosed with muscle wasting or has been diagnosed with being at risk of muscle wasting. Such muscle wasting can be caused by cancer, obesity, metabolic dysfunction, aging, age-related sarcopenia, and/or disuse atrophy.

The T1R2 targeted by the methods disclosed herein can be in any cell comprising T1R2. For example, it can be found in the mouth, brain, heart, kidney, bladder, and nasal respiratory epithelium. Specifically disclosed herein is targeting T1R2 found in skeletal muscle cells. The methods disclosed herein can specifically target T1R2 found in muscle cells only, or can be generally applicable to any T1R2 found in the subject. In some examples, T1R2 is given directly to muscular cells, or targeted specifically to muscle, so that it does not affect T1R2 in other locations such as the mouth. In other examples, a modulator of T1R2 is specific for muscle cells, so that it can be given in any format, but will not affect T1R2 in other locations in the body.

Any agent known to modulate T1R2 can be used with the methods disclosed herein. Examples include, but are not limited to, small molecules which are antagonists or inverse agonist derived from or structurally related to sucrose, glucose, sucralose, saccharin, aspartame, neotame, brazzein, miraculin, S-819, perillartine, P-4000, SE-1, SE-2 (FEMA 4669), SE-3, SE-4, amiloride or gurmarin. In one example, the small molecule can be an antagonist or inverse agonist derived from or structurally related to tas1r2 ligands.

The modulator can also be a nucleic acid inhibitor. For example, such inhibitor can be small interfering RNA (siRNA). Examples of nucleic acid inhibitors include sc-40196 (available from Santa Cruz Biotechnology) and shRNA CAT #: TL505429V (available from Origene). Said nucleic acid inhibition can be accomplished through clustered regularly interspaced short palindromic repeats (CRISPR) technology aiming to develop loss-of-function mutations on T1R2.

According to the methods taught herein, the subject is administered an effective amount of the agent to modulate T1R2. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a T1R2 inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Methods for Identifying Modulators of T1R2

Also disclosed herein is a method of identifying a modulator of T1 R2 in skeletal muscle, the method comprising providing muscle-related cells, exposing the cells to a potential modulator of T1 R2, and determining modulation, thereby identifying a modulator of T1R2. Many aspects of cell physiology can be monitored to assess the effect of ligand binding T1R2. These assays may be performed on intact cells expressing a chemosensory receptor, on permeabilized cells, or on membrane fractions produced by standard methods.

Modulation can be detected by monitoring intracellular NAD levels or PARP-1 activity levels, or other elements which are associated with T1R2 function in muscle cells. For example, Putt et al. disclose screening methods for detecting PARP and NAD quantification (Putt K S, Hergenrother P J. An enzymatic assay for poly(ADP-ribose) polymerase-1 (PARP-1) via the chemical quantitation of NAD(+): application to the high-throughput screening of small molecules as potential inhibitors. Anal Biochem. 2004 Mar. 1; 326(1):78-86. doi: 10.1016/j.ab.2003.11.015, herein incorporated by reference in its entirety).

T1R2 proteins disclosed herein for use in screening methods can be either recombinant or naturally occurring. Examples of screens of T1R2 using modified T1R receptors can be found, for example, in U.S. Pat. No. 8,809,000, herein incorporated by reference in its entirety. The T1R2 proteins or polypeptides can be isolated, co-expressed in a cell, co-expressed in a membrane derived from a cell, co-expressed in tissue or in an animal, either recombinant or naturally occurring. For example, muscle slices, dissociated cells from muscle, stem cells (myoblasts) or transformed cells can be used.

T1R2 modulation can be examined in vitro with soluble or solid state reactions. Ligand binding to T1R2 polypeptides can be tested in solution, in a bilayer membrane, optionally attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

Receptor-G protein interactions can also be examined. For example, binding of the G protein to the receptor complex, or its release from the receptor complex can be examined. More particularly, in the absence of GTP, an activator will lead to the formation of a tight complex of a G protein (all three subunits) with the receptor. This complex can be detected in a variety of ways, as noted above. Such an assay can be modified to search for inhibitors, e.g., by adding an activator to the receptor and G protein in the absence of GTP, which form a tight complex, and then screen for inhibitors by looking at dissociation of the receptor-G protein complex. In the presence of GTP, release of the alpha subunit of the G protein from the other two G protein subunits serves as a criterion of activation. An activated or inhibited G protein will in turn alter the properties of target enzymes, channels, and other effector proteins.

In another embodiment, Fluorescence Polarization (“FP”) based assays may be used to detect and monitor ligand binding. Fluorescence polarization is a versatile laboratory technique for measuring equilibrium binding, nucleic acid hybridization, and enzymatic activity. Fluorescence polarization assays are homogeneous in that they do not require a separation step such as centrifugation, filtration, chromatography, precipitation, or electrophoresis. These assays are done in real time, directly in solution and do not require an immobilized phase. Polarization values can be measured repeatedly and after the addition of reagents since measuring the polarization is rapid and does not destroy the sample. Generally, this technique can be used to measure polarization values of fluorophores from low picomolar to micromolar levels.

Also disclosed herein are soluble assays using a T1R2 polypeptide complex; or a cell or tissue co-expressing T1R2 polypeptides. In another embodiment, disclosed are solid phase based in vitro assays in a high throughput format, where the T1R2 polypeptides, or cell or tissue expressing the T1R2 polypeptides, are attached to a solid phase substrate.

In the high throughput assays of the invention, it is possible to large amounts of potential modulators or ligands in a single day. In particular, each well of a microtiter plate can be used to run a separate assay against a selected potential modulator. Microfluidic approaches to reagent manipulation have been developed.

The molecule of interest (potential modulator) can be bound to the solid state component, directly or indirectly, via covalent or non-covalent linkage, e.g., via a tag. The tag can be any of a variety of components. In general, a molecule which binds the tag (a tag binder) is fixed to a solid support, and the tagged molecule of interest (e.g., the taste transduction molecule of interest) is attached to the solid support by interaction of the tag and the tag binder.

In another embodiment, T1R2 proteins or polypeptides are co-expressed in a eukaryotic cell as chimeric receptors with a heterologous, chaperone sequence that facilitates its maturation and targeting through the secretory pathway. Such chimeric T1R polypeptides can be expressed in any eukaryotic cell, such as HEK-293 cells.

T1R2 modulation may be assayed by comparing the response of T1R2 polypeptides treated with a putative T12R modulator to the response of an untreated control sample. Such putative T1R2 modulators can include potential modulators that either inhibit or activate T1R2 polypeptide activity. In one embodiment, control samples (untreated with activators or inhibitors) are assigned a relative T1R2 activity value of 100. Inhibition of a T12R polypeptide is achieved when the T12R activity value relative to the control is about 90%, optionally 50%, optionally 25-0%. Activation of a T1R2 polypeptide is achieved when the T1R activity value relative to the control is 110%, optionally 150%, 200-500%, or 1000-2000%.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca2+, IP3, cGMP, or cAMP.

T1R2 polypeptide activity can be measured by co-expressing T1R2 genes in a heterologous cell with a promiscuous G protein that links the receptor to a phospholipase C signal transduction pathway (see Offermanns & Simon, J. Biol. Chem., 270:15175-15180 (1995)). Optionally the cell line is HEK-293 (which does not naturally express T1R genes) and the promiscuous G protein is Gα15 (Offermanns & Simon, supra). Modulation of taste transduction is assayed by measuring changes in intracellular Ca2+ levels, which change in response to modulation of the T1R signal transduction pathway via administration of a molecule that associates with T1R polypeptides. Changes in Ca2+ levels are optionally measured using fluorescent Ca2+ indicator dyes and fluorometric imaging.

In one embodiment, the changes in intracellular cAMP or cGMP can be measured using immunoassays. The method described in Offermanns & Simon, J. Bio. Chem., 270:15175-15180 (1995), may be used to determine the level of cAMP. Also, the method described in Felley-Bosco et al., Am. J. Resp. Cell and Mol. Biol., 11:159-164 (1994), may be used to determine the level of cGMP. Further, an assay kit for measuring cAMP and/or cGMP is described in U.S. Pat. No. 4,115,538, herein incorporated by reference.

In another embodiment, transcription levels can be measured to assess the effects of a test compound on signal transduction. A host cell containing T1R2 polypeptides of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays.

The amount of transcription is then compared to the amount of transcription in either the same cell in the absence of the test compound, or it may be compared with the amount of transcription in a substantially identical cell that lacks the T1R2 polypeptide of interest. A substantially identical cell may be derived from the same cells from which the recombinant cell was prepared but which had not been modified by introduction of heterologous DNA. Any difference in the amount of transcription indicates that the test compound has in some manner altered the activity of the TIR polypeptides of interest.

Non-human animals expressing one or more T1R2 polypeptides of the invention, can also be used for receptor assays. Such expression can be used to determine whether a test compound specifically binds to a mammalian taste transmembrane receptor complex in vivo by contacting a non-human animal stably or transiently transfected with nucleic acids encoding chemosensory receptors or ligand-binding regions thereof with a test compound and determining whether the animal reacts to the test compound by specifically binding to the receptor polypeptide complex.

Animals transfected or infected with the vectors of the invention are particularly useful for assays to identify and characterize potential modulators that can bind to a specific or sets of receptors. Such vector-infected animals expressing human chemosensory receptor sequences can be used for in vivo screening of modulators and their effect on, e.g., cell physiology.

Means to infect/express the nucleic acids and vectors, either individually or as libraries, are well known in the art. A variety of individual cell, organ, or whole animal parameters can be measured by a variety of means. The TIR sequences of the invention can be for example co-expressed in animal taste tissues by delivery with an infecting agent, e.g., adenovirus expression vector.

The endogenous chemosensory receptor genes can remain functional and wild-type (native) activity can still be present. In other situations, where it is desirable that all chemosensory receptor activity is by the introduced exogenous hybrid receptor, use of a knockout line is preferred. Methods for the construction of non-human transgenic animals, particularly transgenic mice, and the selection and preparation of recombinant constructs for generating transformed cells are well known in the art.

Construction of a “knockout” cell and animal is based on the premise that the level of expression of a particular gene in a mammalian cell can be decreased or completely abrogated by introducing into the genome a new DNA sequence that serves to interrupt some portion of the DNA sequence of the gene to be suppressed. Also, “gene trap insertion” can be used to disrupt a host gene, and mouse embryonic stem (ES) cells can be used to produce knockout transgenic animals (see, e.g., Holzschu, Transgenic Res 6:97-106 (1997)). The insertion of the exogenous is typically by homologous recombination between complementary nucleic acid sequences. The exogenous sequence is some portion of the target gene to be modified, such as exonic, intronic or transcriptional regulatory sequences, or any genomic sequence which is able to affect the level of the target gene's expression; or a combination thereof. Gene targeting via homologous recombination in pluripotential embryonic stem cells allows one to modify precisely the genomic sequence of interest. Any technique can be used to create, screen for, propagate, a knockout animal, e.g., see Bijvoet, Hum. Mol. Genet. 7:53-62 (1998); Moreadith, J. Mol. Med. 75:208-216 (1997); Tojo, Cytotechnology 19:161-165 (1995); Mudgett, Methods Mol. Biol. 48:167-184 (1995); Longo, Transgenic Res. 6:321-328 (1997); U.S. Pat. Nos. 5,616,491; 5,464,764; 5,631,153; 5,487,992; 5,627,059; 5,272,071; WO 91/09955; WO93/09222; WO 96/29411; WO 95/31560; WO 91/12650.

The nucleic acids of the invention can also be used as reagents to produce “knockout” human cells and their progeny. Likewise, the nucleic acids of the invention can also be used as reagents to produce “knock-ins” in mice. The human or rat TIR gene sequences can replace the orthologous T1R in the mouse genome. In this way, a mouse expressing a human or rat T1R is produced. This mouse can then be used to analyze the function of human or rat T1Rs, and to identify ligands for such T1Rs.

Also disclosed are modulators which are identified using the methods disclosed herein.

### EXAMPLES

### Example 1: Role of T1R2 Signaling Network in the Regulation of Muscle Bioenergetics and Function

Male and female mice are used. If sexual dimorphism is noted in measured variables, experimental sample size is increased and further characterized with separate analysis. Groups of aged-matched 10 to 16-week-old mice (n=8-12 mice/group, gender balanced) are used, except for postnatal muscle growth experiments. It provides adequate power (80%) to generate statistically significant differences in measured variables. Data collection and processing are performed blinded based on mouse or tissue IDs only. The timing of in vivo experiments, tissue and blood collection are matched to avoid diurnal effects. All tissue collections are performed in anesthetized mice using pentobarbital sodium injection to preserve metabolites. Data are analyzed using statistical software with biostatistician assistance, if needed.

Diet: All weaned mice are maintained on standard chow diet (Teklad #7012).

Genetic mouse models: STR signaling requires the obligate heterodimerization of T1R2 and T1R3 proteins (T1R2/T1R3) (49). Thus, ablation of either T1R2, or T1R3 is adequate to eliminate “sweet taste”. Nonetheless, only the T1R2 receptor provides specificity for “sugar sensing”, since the T1R3 receptor also contributes to amino acid (umami) sensing by forming a heterodimer with T1R1 (T1R1/T1R3) (50).

Consequently, mice were made with a) a floxed allele for t1r2 (T1R2^{fl/fl}; KOMP Repository #CSD25803) and b) a targeted Cre knock-in (KI) expressed under the native T1r2 promoter (T1r2-Cre). The Cre gene was inserted at exon 2 and 3 of the T1r2 gene leading to a subsequent stop codon (Ingenious targeting lab, NY). These mice have been crossed with Cre and reporter lines to generate genetic mouse models and littermate controls for these studies: 1) mT/mnG^{T1r2}: T1r2-Cre crossed with ROSA-dual membrane tdTomato/EGFP (mT/nG) (The Jackson Lab 007576) to identify Tr2+ cell lineage (green). 2) T1R2-KO^{Myog}: T1R2^{fl/fl} crossed with Myogenin-Cre mice for constitutive deletion of T1R2 in skeletal muscle. 3) T1R2-KO^{HSA}: T1R2^{fl/fl} crossed with HSA-Cre/Esr1 mice (The Jackson Lab 025750) for conditional (time-dependent) deletion of T1R2 in skeletal muscle. Mice are back crossed on the C57B1\6J strain for at least 10 generations. T1R2-KO (or T1R2-KO^{Myog} male and female mice were used. Figures show mean+SEM, unless otherwise stated.

Previous studies showed that WT and T1R2-KO mice have similar levels of plasma glucose in the fed state (10) and equal responses during an intra-peritoneal glucose tolerance test (IP.GTT) (9, 10), insulin tolerance test (ITT) (12) or a hyperglycemic clamp (10), showing that STRs do not spontaneously affect glucose homeostasis and insulin sensitivity during fed/hyperglycemic conditions. However, it was noticed that T1R2-KO mice have reduced plasma glucose after short-term (5 h) fasting (9, 10). To test whether the mild fasting hypoglycemia was due to increased peripheral glucose disposal, the rate of glucose appearance (Ra) and disappearance (Rd) was monitored using radioisotope enrichment studies ([3-³H]-glucose and 2-deoxy[¹⁴C]glucose) in conscious mice that transitioned undisturbed from ad lib (fed) to short-term fasting (up to 5 h) without insulin infusion (51).

Indeed, both Ra and Rd were significantly increased in T1R2-KO mice during short-term fasting (FIG. 1A). The augmented Rd was due to enhanced glucose uptake specifically in the skeletal muscles of T1R2-KO mice (FIG. 1B) and was accompanied by accelerated depletion of liver glycogen (FIG. 1C). Liver glycogen determines Ra because it is the main contributor to endogenous glucose production during fasting. Critically, no differences between genotypes were noted in a) glucose uptake in the adipose, heart or brain tissue, b) muscle glycogen content, c) plasma insulin or glucagon levels (FIG. 1E/F), and d) GLUT1 and GLUT4 protein levels in isolated T-tubule membranes (FIG. 1G) (52). However, a significant mRNA upregulation of hexokinase II (HXK2) (FIG. 1D) was found, which is the rate-limiting enzyme for glucose utilization and its expression can be induced by glucose flux alone (53). It appears that STRs regulate non-insulin mediated glucose uptake in the skeletal muscle.

To test whether the increased glucose uptake alters glucose utilization and/or skeletal muscle bioenergetics, quantitative targeted metabolite profiling in gastrocnemius muscle of WT and T1R2-KO mice was found after moderate fasting (5 hours) using LC/MC (Southeast Center for Integrated Metabolomics; U24DK097209). Multivariate analyses using discriminative variable selection revealed distinct clustering of metabolite profiles between WT and T1R2-KO muscles (FIG. 2A). Subsequent pathway analysis showed that nicotinamide (i.e. NAD), pyrimidine and purine biosynthesis is significantly upregulated (p<0.00l) in fasted T1R2-KO muscles (FIG. 2B). Particularly, among other metabolites, there were notable increases in the concentrations of NAD*, ATP, GTP, UTP, and CTP (FIG. 2C). No differences in tricarboxylic acid (TCA) cycle intermediates were noted. The increased nucleotide biosynthesis in T1R2-KO muscle was independently confirmed through the modestly elevated levels of intramuscular uric acid (FIG. 2D), indicative of increased purine metabolism (54). Because glucose can be used by the PPP to synthesize nucleotides, NADPH was measured as a readout for PPP activity and found its levels notably increased in T1R2-KO muscles (FIG. 2E). Cellular NADPH is generated from NADP⁺ by glucose-6-phosphate dehydrogenase (G6PD), the rate limiting step in the PPP (55). These metabolic adaptions in T1R2-KO muscles were not accompanied by differences in the expression of key regulators of relevant metabolic pathways (FIG. 3A), suggesting that STR signaling regulates these processes at the enzyme-level. Taken together, this profile can reasonably explain the increased glucose uptake in the skeletal muscle of T1R2-KO mice which, beyond its oxidation, is preferably shunted through the PPP to supply the ribose moiety required for purine and pyrimidine biosynthesis (FIG. 2F). Thus, activation of STR signaling can limit the use of intracellular glucose towards the PPP. This observation shows a striking novel mechanism by which muscle cells can tune the fate of intracellular glucose towards oxidation or biosynthetic processes through direct surveying of extracellular glucose concentrations. It therefore appears that STR-mediated glucose sensing contributes to the regulation of biosynthetic intermediates in skeletal muscle.

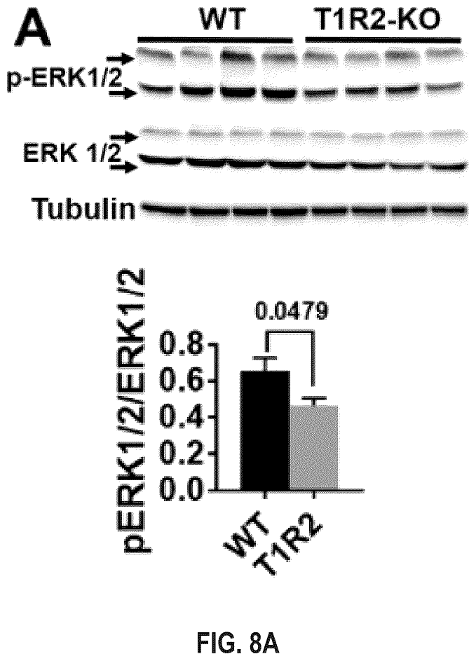

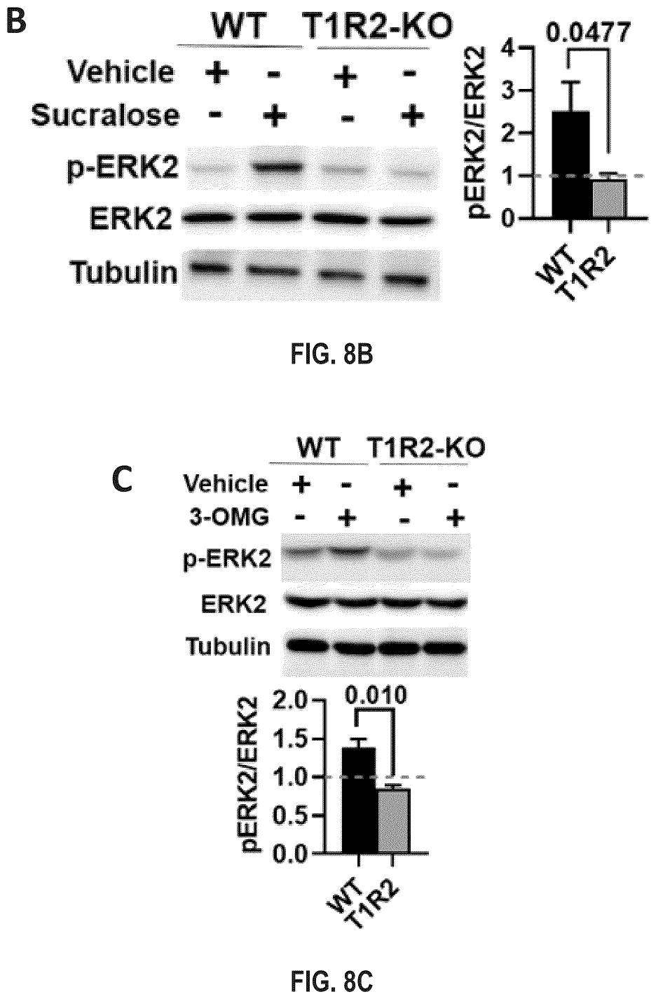

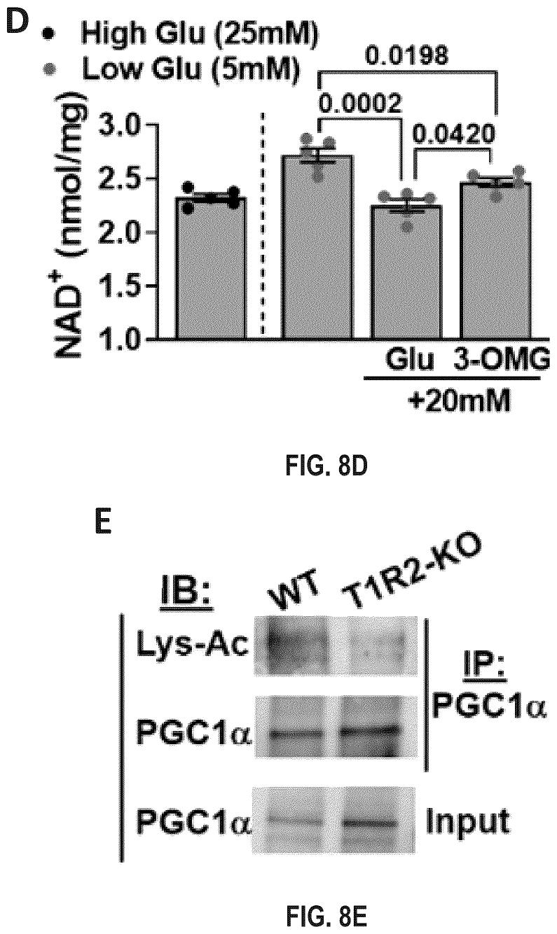

Notably, the most profound observation in fasted T1R2-KO muscles was the extreme depletion (95%) of ADP-ribose (ADPr), which is generated through NAD⁺ consumption (FIG. 2C). ADPr is converted to PRPP, which can be used either for NAD⁺ regeneration (salvage pathway), or for nucleotide synthesis (FIG. 2F), showing that ADPr can be a metabolic link between NAD⁺ and purine/pyrimidine metabolism. Therefore, it was reasoned that the unprecedented depletion of ADPr in T1R2-KO muscles can be due to reduced NAD-consumption and/or increased PRPP utilization for nucleotide biosynthesis. This cam explain the elevated levels of NAD⁺ because no differences were found in the mRNA expression and protein levels of nicotinamide phospho ribosyl transferase (NAMPT), the rate-limiting enzyme in NAD⁺ synthesis (FIG. 3A, B). NAD is mainly consumed by SIRTs, as a cofactor for the deacetylation of proteins, and PARPs, which consume NAD⁺ during the poly(ADP)-ribosylation (PAR) of proteins. Because PARP1 accounts for about 30-50% of cellular NAD⁺ consumption (56), its activity (PAR) was assessed and it was found that it dramatically suppressed in T1R2-KO skeletal muscle (FIG. 3B), but not in other tissues such as the heart or liver, showing that STR singling in the skeletal muscle may regulate PARP1. Inhibition of PARP1 boosts NAD⁺ availability for SIRT activation leading to downstream beneficial metabolic effects, such as improvements in mitochondrial and muscle function (5, 6). So, it was tested whether T1R2-KO muscles have phenotypic similarities to PARP1 inhibition in the muscle. Indeed, high resolution respirometry (HRR) was performed in permeabilized “white” fibers from T1R2-KO gastrocnemius and found higher maximum respiration in the coupled state (O2k respirometer, Oroboros Instruments, Austria) (FIG. 4A). Similarly, the oxidative capacity of T1R2-KO differentiated myoblasts was also increased (XF96 extracellular flux analyzer; Seahorse Bioscience, North Billerica, MA) (FIG. 4B). This phenotype is consistent with the reduced levels of intracellular lactate and increased ATP/ADP ratio in T1R2-KO muscles (FIG. 4C), indicative of enhanced oxidative metabolism (57). The enhanced oxidative profile of T1R2-KO muscles was accompanied by increased succinate dehydrogenase (SDH) staining (FIG. 4D) and increased protein content of respiratory chain complex (OXPHOS) (FIG. 4E), which was not due to an upregulation of OXPHOS genes expression. In addition, the genotype effects on mitochondria function were not due to differences in mitochondrial DNA (mtDNA) content (i.e. copy number), or the expression of genes involved in mitochondrial biogenesis (FIG. 8D).

Consistent with the improvements in muscle bioenergetics and mitochondria function, it was found that T1R2-KO mice were also resistant to exercise fatigue compared to their WT counterparts (FIG. 4F). In addition, using indirect calorimetry it was observed that T1R2-KO mice had increased exercise efficiency during a sub-maximal exercise bout of moderate intensity evident by the lower O₂ consumption and respiratory exchange ratio (RER; VCO₂/VO₂) at steady-state (FIG. 5G, H). Taken together, these findings in T1R2-KO mice recapitulate responses seen during PARP1 inhibition and subsequent activation of SIRT1 (5, 6). Thus, in WT mice activation of STRs, due to rising glucose (fed state), may limit NAD⁺ availability through PARP1 activation, while attenuation of STR signaling, due to declining glucose (as fasting progresses), contributes to NAD⁺ increases (as shown in FIG. 8D below). Strikingly, this mechanism is aligned with known metabolic adaptions of NAD⁺ that take place during fasting and feeding (1, 18), but specific contributions by STRs have never been considered or addressed. It is concluded that STR signaling regulates muscle fitness though NAD*-dependent mechanisms.

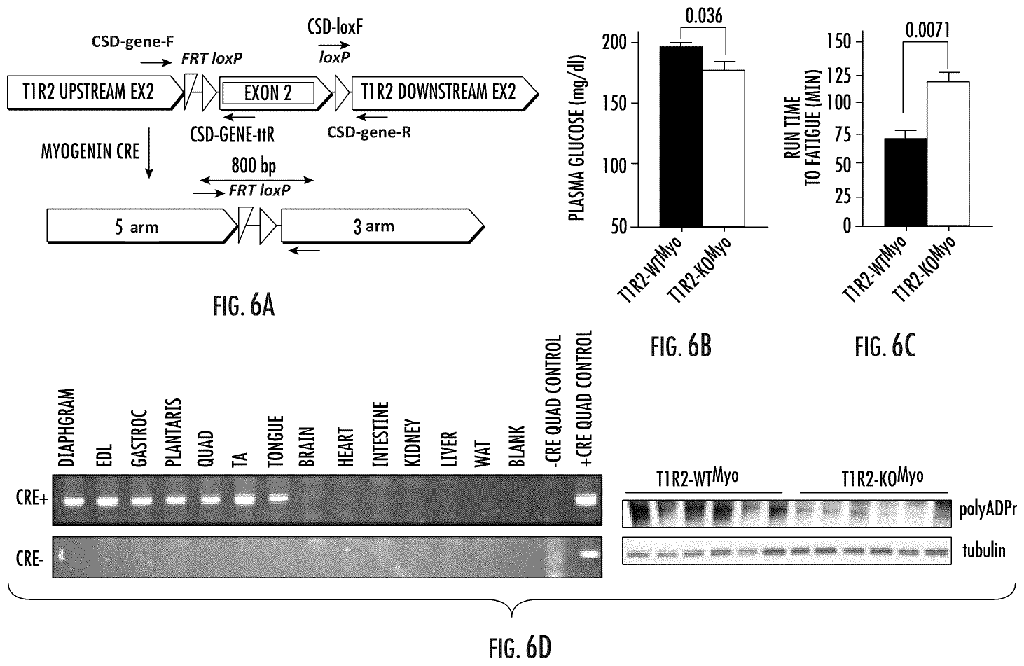

T1r2 and T1r3 are expressed in mouse skeletal muscles (FIG. 5A) and in primary differentiated mouse myoblasts (FIG. 5B) and human myotubes (FIG. 5C). T1r2 was not expressed in cultured myotubes from T1R2-KO mice, confirming STR expression specificity (FIG. 5B). To establish the direct role of STR signaling in the skeletal muscle, mice were developed and characterized with muscle-specific ablation of T1R2 (FIG. 6A). T1R2-KO^{Myog} mice have reduced fasting plasma glucose (FIG. 6B), are resistant to exercise fatigue (FIG. 6C) and have reduced PARP1 activity in the skeletal muscle (FIG. 6D) compared to T1R2-WT^{Myo} controls. In addition, similar to the T1R2-KO phenotype, no differences between T1R2-KO^{Myog} and controls were noted in IP.GTT or ITT responses, mitochondria number, and gene expression profile. Thus, muscle-specific T1R2-KO^{Myog} mice recapitulate key phenotypic outcomes of T1R2-KO mice, supporting a muscle-autonomous role for STR signaling.

Experimental design: The purpose of this aim is to decipher STR-mediated signaling mechanisms in skeletal muscle and explore their interactions with established intracellular sensory pathways. Genetic models: muscle-specific T1R2-KO^{Myog} mice with littermate controls.

The core approach and objectives are implemented using one or more of the following systems: a) in vivo treatments in mice followed by muscle harvesting; b) ex vivo treatments in isolated muscles using specialized oxygenated chambers with temperature and pH control (Physiological Instruments, Inc); c) in vitro treatments in differentiated myoblasts derived from T1R2-KO^{Myog} mice and controls. This primary culture system is more robust and physiologically relevant than cell-lines (i.e. C2C12). However, C2C12 cells are used for overexpression or knockdown of key signaling mediators using lentivirus.

Core approach: a) For in vitro and ex vivo studies the levels of ambient glucose in the media (5-20 mM) were mediated for various times (short-term 1-6 h and/or long-term 24-48 h) to stimulate STR signaling and mimic fed and fasting states. b) For in vivo studies overnight fasting and refeeding were used (2 hours following fasting) to induce substantial fluctuations in circulating glucose. c) For in vivo pharmacological treatments mice are fasted for 5 hours to reduce variations in plasma glucose. The gastrocnemius or quadriceps (mixed fiber type) are studied. However, key findings are also performed in the soleus (oxidative, more Type I fiber) and EDL (glycolytic, more Type II fiber) to account for potential fiber-type specificity. In select in vitro and ex vivo experiments, STRs are further modulated using: i) 3-O-methylglucose (3-OMG), a non-metabolizable glucose analogue (58) that has similar affinity for STRs (59) (also see FIGS. 8C-D). This approach can help to uncouple STR stimulation from the metabolic effects of glucose; ii) sucralose, an artificial sweetener and bonafide ligand for STRs; iii) Gurmarin, a potent and specific pharmacological inhibitor of mouse STRs (60, 61). Combinations of the above core approach are used, as needed, to address the following specific objectives:

Elucidation of signaling pathway leading to PARP1 regulation and NAD⁺ bioavailability. The reduced PARP1 activity in T1R2-KO muscles correlates with substantial increases in NAD⁺ levels (FIGS. 2C and 3B). STR belong to the G_{q} family of GPCRs and, in various tissues, activate phospholipase C (PLC)-mediated signaling cascades (9, 62) (FIG. 7). PARP1 is mainly regulated in response to DNA damage (63), but it was reasoned that a downstream effector(s) of PLC-dependent pathway may regulate PARP1 in skeletal muscle. The focus was on extracellular signal-regulated protein kinases 2 (ERK2) because it can phosphorylate to activate PARP1 independent of DNA damage (64-66) and it is also a plausible effector of the STR/PLC cascade (67). ERK1/2 phosphorylation was reduced in muscles lacking STR signaling (FIG. 8A), while intra-muscular injection of potent STR ligands, such as sucralose (FIG. 8B) or 3-OMG, a non-metabolizable glucose analogue (FIG. 8C), specifically activated ERK2 phosphorylation in WT, but not in T1R2-KO muscles.

Specific approach: The general strategy is to manipulate common mediators of the Go-GPCR (i.e. STR) and mitogen-activated protein kinase (MAPK)/ERK signaling in the context of specific STR stimulation. The PLC/PKC/c-RAF/ERK2 is targeted (PKC, protein kinase C) cascade, because this is also consistent with PARP1 activation in other cell-types (66). Briefly, PLC inhibition (U73122) was used to achieve upstream blockade of STR signaling (9), and c-RAF inhibition (CNI-1493, semapimod) as a PKC-mediated downstream target and ERK1/2 activator. To further establish causality, key identified mediators are knocked down using stable lentivirus shRNA in C2C12 and/or primary cultures. a) ERK2 specific PARP1 phosphorylation (64) and activity (auto-PARylation as in FIG. 3B) in nuclear and cytoplasmic fractions, b) phosphorylation of MAPK/ERK2 cascade (i.e. c-RAF/MEK-1/2/ERK1/2), c) concentrations of NAD*and its metabolic intermediates (NAM, ADPr, O-acetyl-ADPr, and cyclicADPr) using LC/MS. To prevent NAD⁺ synthesis or PAR hydrolysis to ADPr, NAMPT (FK866) (3) and PAR glycohydrolase are inhibited (PARG; GPI18214) (68), respectively (FIG. 3F) are used.

Determination of downstream effectors of NAD⁺-SIRT axis. To shed light on the link between STR activation and NAD regulation, differentiated C2C12 cells were cultured at high (25 mM) or low (5 mM) glucose for 54 hours and observed an anticipated increase in NAD levels during the low-glucose condition (FIG. 8D) (18, 69). Addition of 20 mM glucose in low-glucose cultured cells during the last 6 h decreased NAD levels back to those seen in high-glucose cultured cells. Strikingly, addition of equal molarity (20 mM) 3-OMG (glucose analogue) also caused a significant drop in NAD, although to a lesser extent than 20 mM glucose (FIG. 8D). Because 3-OMG has equal affinity for STRs as glucose (59) but it cannot be metabolized by cells (58), the data suggest that the suppressing effects of high glucose on NAD levels were partially mediated by STR activation. Thus, it appears that a STR-mediated regulation of NAD⁺-SIRT axis is sufficient to account for key muscle adaptations in T1R2-KO mice. There are seven sirtuins (SIRT1-7), but SIRT1 and SIRT2 were identified as the most plausible candidates. This is based on the muscle-specific phenotype of T1R2-KO mice, the physiological effects of SIRTs, their tissue expression and subcellular localization, and their affinity for NAD⁺. SIRT1: Inhibition of PARP1 has direct effects on nuclear NAD levels which significantly impacts SIRT1 activation (70) due to substrate competition (NAD⁺ Kₘ, PARP1: 50-95 M vs. SIRT1: 95 M) (1). There are numerous SIRT1 targets (FOXO1/3, NF-κB, CREB, TORC2, LXR, MyoD, MEF2, SREBP-1 and BMAL1) with functions relevant to metabolism (71), but SIRT1-mediated deacetylation of peroxisome proliferator activated receptor-γ coactivator-1α (PGC1α) induces similar mitochondrial and exercise adaptations seen in T1R2-KO mice (72) (FIG. 4). PGC1α protein levels or mRNA expression were not different in T1R2-KO muscles but it was found reduced PGC1α acetylation in gastrocnemius muscle, suggesting increased SIRT1 activity (FIG. 8E). These data support the involvement of a NAD⁺-SIRT1-PGC1α regulatory axis in STR-induced signaling cascade.

Specific approach (SIRT1): Selective inhibition of SIRT1 (Ex-527) (73-75) and PGC1α (SR-18292) (76) activity in the context of STR activation and in combination with select mediators of STR-PARP1 cascade are performed. SR-18292 specifically and rapidly increases PGC1a lysine acetylation independently from SIRT1 or other histone deacetylases (HDAC). To further establish causality, PARP1 or SIRT1 were knocked down using shRNA in C2C12 and/or primary cultures (77). Access was: a) PGC1α lysine acetylation using acetyl-lysine antibody (Cell Signaling) following immunoprecipitation (IP) of PGC1α (Millipore) (FIG. 8E) (78), b) SIRT1-PGC1α-regulated transcriptional targets (77), c) mitochondrial function: i) HHR in intact permeabilized fibers (Oroboros; FIG. 4A (79)), ii) citrate synthase activity in muscle homogenates (Millipore Sigma), iii) SDH (FIG. 4D) and cytochrome oxidase (COX) staining in muscle section, iv) mitochondria mtDNA and protein content. SIRT2: Unlike SIRT1, the role of SIRT2 in the skeletal muscle is not well defined, but it has been linked to insulin resistance (80) and insulin-sensitive glucose transport (81). Although these functions are not directly aligned with the T1R2-KO phenotype, SIRT2 can affect the PPP through the acetylation of G6DH (82-84). This is consistent with the increased G6PD activity (elevated NADPH levels) and nucleic acid biosynthesis in T1R2-KO muscles (FIG. 4), which cannot be explained by differences in SIRT2 or G6PD1 gene expression FIG. 3A). Notably, SIRT2 has very similar affinity for NAD⁺ as SIRT1 (Km SIRT1: 95 μM, SIRT2: 83 μM) (1), showing that PARP1 inhibition can potentially affect both enzymes. Although SIRT2 and G6DH are localized in the cytoplasm, the NAD⁺ precursor, NAM nucleotide (NMN) can cross intracellular membranes leading to NAD⁺ exchange between the nucleus and cytoplasm (56, 85).

Specific approach (SIRT2): SIRT2 is selectively inhibited with thiomyristoyl (TM) (86) in the context of STR signaling (i.e. ERK2/PARP1). To further establish causality, SIRT2 was knocked down using shRNA in C2C12 and/or primary cultures. Access was: a) G6DH lysine acetylation following IP of G6PD, b) G6DH activity and assessment of NADPH/NADP⁺, c) nucleic acid concentrations using LC/MS, d) expression of key enzymes in PPP and nucleotide biosynthesis (85), e) ex vivo basal glucose uptake to establish the link between glucose uptake and PPP/nucleic acid biosynthesis (also see Aim1.c), f) and finally, because NADPH counteracts oxidative damage through reduced glutathione (GSH) formation, the link between enhanced NADPH in T1R2-KO mice and resistance to oxidative stress was tested using H₂O₂ or dithiothreitol (DTT) and assess GSH/GSSG (reduced/oxidized) and total glutathione (Biovision #K264).

Contribution of STR signaling in the regulation of glucose utilization. The increased glucose flux in the skeletal muscle of T1R2-KO mice is consistent with the enhanced oxidative capacity (FIGS. 1 and 4) and the increased whole-body carbohydrate utilization previously reported (12). Thus, STR-mediated glucose sensing may also regulate substrate oxidation in the skeletal muscle.

Specific approach: Relevant components of STR signaling were manipulated to assess their role in the regulation of glucose uptake and oxidation. The focus was primarily on the ERK2/PARP1/NAD⁺/SIRT2 axis since SIRT2 may regulate PPP and thus glucose flux through that route. Assessment was: a) ex vivo basal or insulin-stimulated [³H]-2-deoxyglucose uptake in isolated intact muscles (EDL and soleus) (87) using customized oxygenated chambers, b) signaling relevant to glucose transport (i.e. insulin, 5′ AMP-activated protein kinase; AMPK), c) plasma membrane GLUT1 and GLUT4 levels (52), d) substrate oxidation in cultured myotubes or muscle homogenates using ¹⁴CO₂ trapping of ¹⁴C-glucose (88), e) HRR in intact fibers in response to glucose (pyruvate) (Oroboros; FIG. 4A) (79).

STR signaling interactions with established intracellular energy sensors. In myocytes, glucose can be sensed by AMPK (89), mammalian target of rapamycin C1 (mTORC1) (90), Akt dependent-signaling and other molecular sensors (14, 32). Signals derived from these pathways often intersect, suggesting that cellular decisions must be justified through the integration of multiple energy cues. Unlike these intracellular sensors which primarily sense glucose-derived metabolites, STRs signaling uniquely delivers energy cues through direct sensing of extracellular glucose. Interestingly, the data show that the enhanced glucose uptake in T1R2-KO muscles is not mediated through insulin-sensitive mechanisms (9, 10, 12). It appears that STR signaling is not directly coupled to, but likely converges downstream with traditional sensory mechanisms and contributes to the regulation of common cellular outcomes (i.e. mitochondria function, biosynthetic potential, substrate utilization).

Specific approach: Pathway interactions were tested in the context of STR stimulation and determine their interdependency. Pharmacological inhibitors/activators are used to manipulate Akt, AMPK or mTORC1 signaling (i.e. AICAR and Compound C for AMPK) and assess key signaling outcomes linked to STRs (ERK2, PARP1, SIRT1,2). Upon identification of potential signaling nodes, their contribution to relevant cellular adaptations is explored (as in FIGS. 2 and 4).

Results: STRs trigger a PLC→(MAPK)→ERK2 cascade (arrows indicate multiple steps). Similar pathways have been demonstrated in skeletal muscle with amino acid receptors (91). ERK2 can regulate PARP1 phosphorylation and activity, which is the main mechanism by which STRs control NAD⁺ bioavailability. Consequently, STR-mediated muscle adaptations are directly linked to NAD-SIRT axis regulation. SIRT1 and SIRT2 can be the main effector sirtuins because together they can account for most STR-related muscle adaptations (glucose utilization and nucleic acid synthesis, mitochondrial function, exercise tolerance etc.). STR signaling likely intersects with other primary mechanisms which may be required for the manifestation of STR-mediated effects. Finally, basal glucose uptake is increased in T1R2-KO^{Myog} muscles. These effects are independent of insulin or AMPK signaling, but linked to increased glucose shunt and utilization through the PPP. Muscles from T1R2-KO^{Myog} mice has enhanced oxidative capacity because of improved mitochondria function.

### Example 2: Contributions of T1R2-Mediated Glucose Sensing in the Regulation of Skeletal Muscle Mass

Rationale: T1R2-KO (12) and T1R2-KO^{Myog} mice (FIG. 9A) have increased lean mass (EchoMRI), so it was assessed whether the augmented pool of biosynthetic intermediates in the muscle (FIG. 2) leads to muscle growth. Indeed, both T1R2-KO and T1R2-KO^{Myog} muscles have mild increases (15-20%) in fiber size (FIGS. 9B and C), confirming the muscle-autonomous effects of STR signaling. No differences in muscle fiber type (FIG. 9D) or myofiber number (soleus; WT: 670±46.97 vs. T1R2: 666±52.35 p=0.95) were noted. To test whether the mild myofiber growth in T1R2-KO mice can prevent/delay muscle mass loss associated with metabolic dysfunction, T1R2-KO mice were crossed with ob/ob mice, a genetic model of obesity that also manifests muscle atrophy (38, 39). Genetic deletion of STR in ob/ob mice (ob/ob^{T1R2-KO}), partially preserved muscle mass losses (FIG. 9E) without affecting fiber type distribution or number and caused improvements in glucose tolerance (IP.GTT) (FIG. 9F) compared to ob/ob^{T1R2-WT}. Similarly, it was shown that T1R2-KO mice fed a HFD for 12 weeks preserved their lean mass compared to their WT counterparts (12). It is therefore plausible that the chronic and sustained hyperglycemia in T2D and obesity may constitutively hyper-activate STR signaling in the skeletal muscle contributing to muscle mass loss. Thus, it appears that receptor-mediated glucose sensing regulates mechanisms that control adaptations in muscle mass. This phenotype suggests a mechanism by which STR signaling can contribute to the regulation of the anabolic potential of skeletal muscle. To further test this possibility, anabolism was suppressed by subjecting mice to an overnight (o/n) fast, followed by 2 hours of refeeding to subsequently jumpstart anabolic processes. As a readout, the protein levels and phosphorylation of eukaryotic translation initiation factor 4E (eIF4E)-binding protein 1 (4E-BP1) and ribosomal protein S6 (rpS6) was assessed which are indicators of protein translation. As expected, rpS6 and 4E-BP1 were induced in both genotypes during refeeding following and o/n fast, but these effects were slightly potentiated in T1R2-KO muscles compared to WT controls (FIG. 10A). Notably, refeeding caused a rapid upregulation of STRs (T1r2 and T1r3) (FIG. 10B). This could potentially further enhance STR function in WT muscles (but not in T1R2-KO mice since both T1R2 and T1R3 are required for functional STRs). Both genotypes had similar plasma glucose levels, lost and regained the same weight, and consumed the same amount of food in response to o/n fasting and feeding. To directly test the effects of enhanced translational capacity on protein synthesis, the surface sensing of translation (SUnSET) method was used (101) and found increased rates of in vivo protein synthesis in T1R2-KO muscles (FIG. 10C). Notably, no differences in the expression of genes (Murf1,2; Mul1; Foxo1,3; Fbxo40; Atrogin1; IL6; Mstn) known to be upregulated during atrophic stimuli, were found, showing that STR signaling may not affect muscle mass through the regulation of catabolic mechanisms. These data may indicate that, in response to ambient glucose levels, STR signaling in WT mice acts as an adaptive “break” to fine tune biosynthesis. However, the permanent deletion of STR signaling in T1R2-KO mice abolishes that “break” priming NAD⁺ and nucleotide levels (FIG. 2). This can lead to enhanced protein synthesis and muscle growth. It appears that STR-mediated glucose sensing links plasma glucose availability to the anabolic potential of the skeletal muscle.

Experimental design: The purpose is to assess functional and molecular adaptations in response to anabolic and catabolic stimuli in adult muscles, delineate interactions between STR-mediated pathways and established molecular mediators that control muscle mass, and explore the role of T1R2 sensor during postnatal muscle growth. Genetic models: T1R2-KO^{Myog} mice for constitutive (embryonic) deletion and T1R2-KO^{HSA} mice for conditional (time-dependent) deletion in the skeletal muscle with appropriate littermate controls. Comparison between constitutive and time-dependent deletion of STRs elucidates which aspects of muscle adaptations require or depend on embryonic, neonatal or adult STR signaling.

Physiological effects of conditional deletion of STR glucose sensing in adult skeletal muscle. To decisively determine whether the regulation of muscle mass is dependent on STR signaling in adult muscles or on altered mechanisms during postnatal muscle growth, muscle mass and function (plasticity) is assessed and compared in T1R2-KO^{Myog} (constitutive deletion) and T1R2-KO^{HSA} (conditional deletion) mice. For conditional deletion, an IP injection of 2 mg/d tamoxifen (tam) is used in twelve-week old T1R2-KO^{HSA} (i.e. Cre⁺) or T1R2-WT^{fl/fl} (i.e. Cre⁻) mice for five consecutive days. Two-weeks following the 5-day treatment, tamT1R2-KO^{HSA} and tamT1R2-WT^{fl/fl} control mice are assigned to experimental groups. This period is adequate for flox recombination (102). The efficacy of this protocol has been validated. Comparisons with vehicle-treated T1R2-KO^{HSA} and with constitutive T1R2-KO^{Myog} mice are age-matched. The outcomes of this approach are important and physiologically relevant because conditional deletion of T1R2 signaling in adult muscles better mimics the potential longitudinal effects of pharmacological treatments targeting STRs.

Specific approach: a) Assessment of in vivo metabolic control: i) body composition (EcoMRI); ii) ITT, and IP.GTT (plasma glucose and insulin); iii) fed and fasted blood (glucose, insulin, triglycerides, cholesterol, fatty acids) iv) energy balance, food intake, ambulatory activity using metabolic cages (Phenomaster, TSE Systems), v) sub-maximum and maximum exercise calorimetry (FIG. 4G-H) (CaloTreadmill, TSE Systems) and exercise endurance (FIG. 4F). b) Assessment of skeletal muscle phenotype and signaling: i) non-invasive in vivo assessment of muscle function (i.e. limb grip strength, muscle contractility and fatigue, motor unit function); ii) morphological characteristics (fiber number, size, type etc.) of representative muscles (soleus, EDL, gastroc); iii) muscle protein synthesis and degradation using standard methodology (SUnSET; growth- and atrophy-related gene expression; ubiquitin content; mRNA of proteasome subunits; markers of autophagy) (103, 104); iv) signaling mediators of growth and atrophy (i.e. mTOR; Akt; FOXO) (105); v) essential components of STR signaling as identified in Example 1. It was elucidated whether ablation of STRs in adult muscles alters fundamental STR signaling outcomes linked to muscle bioenergetics and mass. c) Mitochondrial function (as in Example 1): i) mitochondrial enzyme level and activity; ii) HRR in isolated mitochondria and intact muscle fibers.

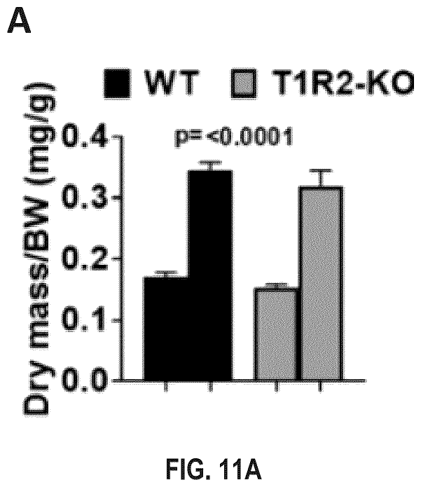

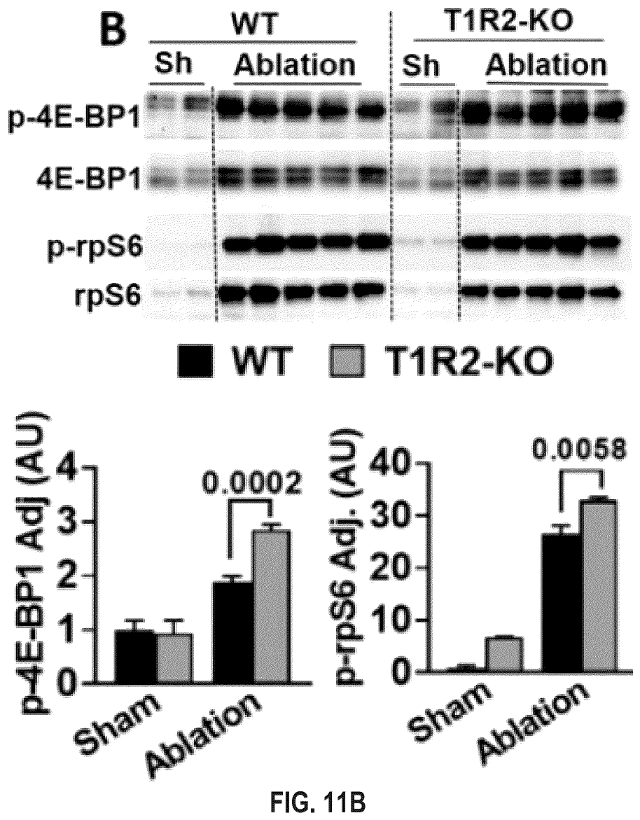

Contributions of STR signaling to muscle mass adaptations. Skeletal muscle mass is regulated through the integration of signals derived from anabolic and catabolic pathways that affect, among other processes, protein turnover (106). STRs deliver to the muscle signals representing peripheral energy status through direct glucose sensing. Thus, the increased levels of nucleic acids and enhanced protein synthesis may be directly linked to the mild myofiber hypertrophy in T1R2-KO and T1R2-KO^{Myog} muscles and account for the partial protection from muscle mass losses in ob/ob^{T1TG2KO} mice (FIG. 9E) and HFD-fed T1R2-KO mice (12). These findings show that strategies aiming to inhibit STRs may decelerate muscle wasting associated with obesity and metabolic dysfunction, but these effects are also likely applicable to other conditions associated with muscle mass wasting, such as aging, bed rest, and disuse atrophy. To test the possibility that deletion of STR signaling would enhance muscle mass gains in response to anabolic stimuli, a pilot study in WT and T1R2-KO mice was performed using synergist ablation (SA) surgery which removes the soleus and distal half of the gastrocnemius and causes hypertrophy of the plantaris (PLA) due to mechanical overload (107). Robust, but similar maximum muscle mass gains of the PLA muscle in WT and T1R2-KO mice was found following 14 days of surgery (FIG. 11A). Nevertheless, a small, but significant, potentiation in the induction of the protein translation markers was found, 4E-BP1 and rpS6 (FIG. 11B).

These findings are not unexpected because ablation of STRs is more likely to prime and/or accelerate anabolic pathways—through nucleotide availability and protein synthesis—than to potentiate absolute or maximum hypertrophic responses. Based on these studies, it appears that deletion of STR signaling can a) prevent/delay muscle mass loss in response to catabolic stimuli, and b) accelerate muscle growth in response to i) anabolic stimulus, or ii) recovery following atrophic stimulus.

Specific approach: Loss-of-function T1R2-KO^{Myog} mice were used to investigate the role of STR signaling in the regulation muscle mass adaptations. T1R2-KO^{HSA} mice are selectively used to test whether the most significant outcomes also persist during STR deletion in adult muscles. There are several conditions/diseases that are associated with muscle mass loss (i.e. starvation, cancer cachexia, aging, diabetes, motor neuron disease) or gain (high intensity exercise training, anabolic hormone stimulation), but the simultaneous occurrence of other systemic alterations accompanying these conditions can convolute muscle-specific mechanisms leading to mass adaptations. Because this is the first attempt to elucidate contributions of STR signaling in muscle biology, established experimental protocols were used that induce rapid and specific changes in muscle mass. Unilateral hind limb immobilization (UHI) was used to induce disuse muscle atrophy and bilateral SA surgery to induced compensatory muscle hypertrophy, because these protocols generate predictable outcomes and several adaptive mechanisms have been described (107, 108). 1) Muscle atrophy: UHI is performed as described (108, 109). The contralateral leg serves as the internal control since it is known there are no changes in muscle mass or function. Mice are used following 5 or 10 days of immobilization. Separate cohorts of mice are first immobilized for 10 days and then the cast is removed to monitor muscle adaptations following 5 and 10 days of recovery. The gastrocnemius and soleus muscles are studied. 2) Muscle hypertrophy: Bilateral SA surgery is performed as described (110) (FIG. 11). Because STR signaling is unlikely to affect maximum growth responses (FIG. 11), mice are studied dynamically and compared to sham controls following 3, 6, 9, 14 days post-SA surgery to assess whether ablation of STRs accelerate hypertrophic responses. Comparisons were started at day 3 following SA surgery to avoid the initial inflammation associated with the procedure. Mice subjected to both protocols are housed individually and food and water are monitored. a) Assess signaling and muscle phenotypic adaptations to capture contributions of STR sensing at different stages of muscle atrophy, recovery following atrophy, or hypertrophy: i) muscle morphology (fiber type and CSA); ii) general markers of translational capacity and efficiency (total and phosphorylated rpS6 and eIF4G, expression of 5.8 s and 18 s rRNA). iii) protein synthesis using SUnSET with immunohistochemistry (muscle sections) and immunoblotting (muscle homogenates) (104, 111); iv) protein degradation using a proteasome activity assay (Proteosome 20S assay kit, Enzo Life Sciences) with or without MG132 (a 20 S proteasome inhibitor), or autophagy (calpain activity) in muscle homogenates; v) signaling and transcriptional control of major positive (i.e. IGF1-PI3K-Akt-mTOR; c-myc; myoD) and negative (i.e. myostatin-Smad2/3; FOXO-MuRF1/MAFbx; AMPK) molecular modulators of muscle mass; vi) Essential components of STR signaling as identified in Example 1; vii) Once STR-mediated signaling contributions to main pathways that control muscle mass adaptations are identified, isolating and evaluating independent effects of STR signaling is done. For instance, ablation of STRs can potentiate mTORC1-induced targets (rp6 and 4E-BP1, FIG. 10A), so inhibition of mTOR (i.e. rapamycin) (112) is evaluated for independent residual effects mediated by STRs; viii) mitochondrial function. ix), assessment of blood hormones that regulate muscle mass (i.e. IGF1; insulin; myostatin). b) Assess the dynamics of functional adaptations during hypertrophy, atrophy, or recovery following atrophy using in vivo assessment of muscle function (i.e. limb grip strength, muscle contractility and fatigue, motor unit function). Non-invasive in vivo functional approaches allow treated mice to be assessed longitudinally (repeated measures) and help explore interactions between functional and corresponding cellular adaptation.

Assessment of spatiotemporal expression of T1R2 during muscle growth. STRs are expressed in adult skeletal muscles (FIG. 5A), but whole muscle includes myocytes and several other cell types, so Myogenin-Cre mice were crossed with RiboTag^{fl/fl} mice (The Jackson Lab 029977), which express the hemagglutinin (HA) epitope targeted ribosomal protein L22 (Rpl22) when bred to Cre-expressing mice (113). Thus, RiboTag^{Myog} mice have Cre-mediated HA epitope tagging of ribosomes from myogenin expressing cells to allow immunoprecipitation of mRNA selectively from myocytes. Compared to whole-muscle input control, there was robust enrichment of Myh2 (muscle-specific marker) and T1r2 expression in mRNA pulled down from adult RiboTag^{Myog} myofibers (FIG. 12A), further confirming that STRs are expressed in myocytes in vivo. In primary cultures, STR expression was very low in undifferentiated myoblasts, but it was progressively increased as myoblast were differentiating to myotubes which can mimic events occurring during embryonic muscle development. That led to also preliminarily assessing STRs expression during postnatal muscle period and a significant upregulation of STR expression (20×) was found in muscles at postnatal day 1 (P1) that was drastically reduced at P7 and P21 to levels seen in adult muscles (FIG. 12B). Thus, it appears that the dynamics of STR expression may be relevant to muscle development and growth because, beyond STR signaling, a decrease in the expression of STRs in early neonatal period would also promote muscle growth. A precise assessment of STR expression profiling in developing muscle can help to elucidate possible mechanistic contributions in the regulation of muscle mass.

Specific approach: A T1r2 gene reporter mouse (mT/mG^{T1r2}) was used to characterize the cell lineage of T1r2 gene expression (FIG. 12C). To specifically assess spatiotemporal T1r2 expression, mT/mG^{T1r2} tissues are used in combination with the co-expression of T1R2 and Cre proteins using commercially available antibodies to identify cells that actively express T1r2 at the time of assessment. To understand the kinetics of T1r2 expression during different stages of muscle development and growth, appropriate antibodies can also be used to identify satellite cells (Pax7), committed myoblasts (MyoD), differentiated myoblasts (Myogenin), fusing myotubes/myofibers (desmin), type I fibers (Myh7), type Ha (Myh2), type IIb (Myh4), embryonic (Myh_{emb}), neonatal (Myhₙₑₒ) and DAPI to identify nuclei. Fluorescent microscopy is complemented with qPCR-based mRNA expression using RiboTag^{Myog} muscles to specifically extract mRNA only from myocytes (i.e. myogenin expressing cells) and to assess the relationship between STR expression and key determinants of muscle development and growth. Specifically, T1r2 co-expression with proteins/genes of interests is assessed (microscopy and qPCR) in: a) embryos (harvested at 12, 15 and 18 days post-coitum); b) postnatal muscles (days P1, P7, P14, P21 and P28); c) adult muscles and isolated fibers, d) satellite cells from isolated single fibers (114); e) Using floating single fibers in culture (115), T1r2 expression was assessed in quiescent (immediately post-harvest) activated (within 24 hour post-harvest) and proliferating (48 h post-harvest, BrdU incorporated) satellite cells; f) and primary myoblasts in vitro (during proliferation and at 2, 4, and 6 days of differentiation).

Contributions of STR-mediated glucose sensing during postnatal muscle growth. Using primary myoblast cultures, similar myofiber morphology and expression of differentiation markers were observed between genotypes (FIG. 12C). These data suggest that ablation of STR may not cause apparent defects in myoblast differentiation in vitro. However, germ line (whole body) or embryonic (muscle-specific myog-Cre) deletion of STRs led to mild increases in muscle mass assessed in adult mice (FIG. 9B, C), without altering the number of fibers per muscle. In mice, early postnatal muscle growth is achieved through the addition of satellite cell-derived new myonuclei, causing hypertrophy of existing myofibers, instead of increases in the number of myofibers (hyperplasia) (116). Because of the differential STR expression in neonatal muscles (FIG. 11B), it was reasoned that STRs may play a role during postnatal muscle growth which could also be linked to the mild increases in fiber size seen in adult (developed) T1R2-KO muscles.

Specific approach: Using T1R2-KO^{Myog} loss-of-function mice it was found that the contributions of STR-mediated glucose sensing during postnatal growth (P7, P14, P21 and P60 for adult). The extensor digitorum longus (EDL) muscle is used because it is easily accessible in postnatal mice, it has uniform type II fiber establishment early during development (117, 118), and the data show that all types of fibers are equally hypertrophied in T1R2-KO muscles (Type I in soleus and Type Ha in gastrocnemius. Assessment included: a) number of fibers per EDL CSA; b) myofiber CSA; c) length of isolated myofibers; d) myofiber volume calculated by mean length multiplied to mean CSA; e) number of myonuclei per myofiber; f) myonuclear domain calculated by myofiber volume divided by mean number of myonuclei per fiber; g) number of satellite cells per single isolated myofiber and per EDL CSA; h) signaling mechanisms relevant to postnatal muscle growth that converge to Akt and mTOR (i.e. IGF-1/PI3K and follistatin through myostatin inhibition), i) rates of intramuscular protein synthesis (SUnSET). It is expected that elimination of STR signaling in adult muscles (T1R2-KO^{HS}n) entirely recapitulates the main metabolic phenotype (i.e. improved mitochondria function, resistance to fatigue, NAD⁺-SIRT axis activation) seen in constitutive STR deletion (T1RR2-KO^{Myog}), but a spontaneous increase in muscle mass size can be less apparent which would suggest a role of STRs during postnatal muscle growth.

Ablation of STRs can accelerate hypertrophic responses following SA surgery suggesting anabolic priming, but it is unlikely to further potentiate absolute hypertrophy. In contrast, deletion of STR signaling can attenuate mass muscle loss associated with UHI and likely accelerate recovery following UHI. These outcomes suggests that reduced STR signaling during low glucose availability (prolonged fasting) enhances biosynthetic pathways to counteract the global muscle catabolism associated with a state of energy deficit. Thus, pharmacological inhibition of STRs can decelerate muscle mass loss that accompanies catabolic conditions (i.e. cachexia, bed rest) or age-associated atrophy. 2.c) STR is expressed during embryonic muscle development and early during postnatal growth (P1<P7) followed by reduced expression until adulthood to potentiate muscle growth. However, STRs may be expressed in satellite cells accounting for the high T1R2 expression seen in P1 muscles. If needed, in addition to myofiber-specific expression profiling (RiboTag^{Myog}) w RiboTag^{fl/fl} can be crossed with Pax7-Cre mice to specifically isolate mRNA from satellite cells. No expression selectivity is anticipated between fiber types, although the expression levels among fiber types may be different.

Based on these findings, ablation of STRs during postnatal growth could cause: a) increased myonuclei fusion and induction of mild muscle hypertrophy that continues to adulthood, or b) normal growth during early postnatal period, but muscle mass can progressively increase due to the increased anabolic intermediates and protein synthesis associated with STR elimination. If the former outcome (a) is observed, T1R2-KO^{HSA} mice can be used for postnatal conditional deletion to dissect temporal STR-mediated contributions in muscle mass.

### REFERENCES

1. Canto C, Menzies K J, Auwerx J. NAD(+) Metabolism and the Control of Energy Homeostasis: A Balancing Act between Mitochondria and the Nucleus. Cell Metab. 2015; 22(1):31-53. Epub 2015/06/30. doi: 10.1016fj.cmet.2015.05.023. PubMed PMID: 26118927; PMCID: PMC4487780.
2. Elhassan Y S, Philp A A, Lavery G G. Targeting NAD⁺ in Metabolic Disease: New Insights Into an Old Molecule. J Endocr Soc. 2017; 1(7):816-35. Epub 2017/12/22. doi: 10.1210/js.2017-00092. PubMed PMID: 29264533; PMCID: PMC5686634.
3. Frederick D W, Loro E, Liu L, Davila A, Jr., Chellappa K, Silverman I M, Quinn W J, 3rd, Gosai S J, Tichy E D, Davis J G, Mourkioti F, Gregory B D, Dellinger R W, Redpath P, Migaud M E, Nakamaru-Ogiso E, Rabinowitz J D, Khurana T S, Baur J A. Loss of NAD Homeostasis Leads to Progressive and Reversible Degeneration of Skeletal Muscle. Cell Metab. 2016; 24(2):269-82. Epub 2016/08/11. doi: 10.1016/j.cmet.2016.07.005. PubMed PMID: 27508874; PMCID: PMC4985182.
4. Ryu D, Zhang H, Ropelle E R, Sorrentino V, Mazala D A, Mouchiroud L, Marshall P L, Campbell M D, Ali A S, Knowels G M, Bellemin S, Iyer S R, Wang X, Gariani K, Sauve A A, Canto C, Conley K E, Walter L, Lovering R M, Chin E R, Jasmin B J, Marcinek D J, Menzies K J, Auwerx J. NAD⁺ repletion improves muscle function in muscular dystrophy and counters global PARylation. Sci Transl Med. 2016; 8(361):361ral39. Epub 2016/11/01. doi: 10.1126/scitranslmed.aaf5504. PubMed PMID: 27798264; PMCID: PMC5535761.
5. Bai P, Canto C, Oudart H, Brunyanszki A, Cen Y, Thomas C, Yamamoto H, Huber A, Kiss B, Houtkooper R H, Schoonjans K, Schreiber V, Sauve A A, Menissier-de Murcia J, Auwerx J. PARP-1 inhibition increases mitochondrial metabolism through SIRT1 activation. Cell Metab. 2011; 13(4):461-8. doi: 10.1016/j.cmet.2011.03.004. PubMed PMID: 21459330; PMCID: PMC3086520.
6. Pirinen E, Canto C, Jo Y S, Morato L, Zhang H, Menzies K J, Williams E G, Mouchiroud L, Moullan N, Hagberg C, Li W, Timmers S, Imhof R, Verbeek J, Pujol A, van Loon B, Viscomi C, Zeviani M, Schrauwen P, Sauve A A, Schoonjans K, Auwerx J. Pharmacological Inhibition of poly(ADP-ribose) polymerases improves fitness and mitochondrial function in skeletal muscle. Cell Metab. 2014; 19(6):1034-41. doi: 10.1016/j.cmet.2014.04.002. PubMed PMID: 24814482; PMCID: PMC4047186.
7. Laffitte A, Neiers F, Briand L. Functional roles of the sweet taste receptor in oral and extraoral tissues. Curr Opin Clin Nutr Metab Care. 2014; 17(4):379-85. doi: 10.1097/MCO.0000000000000058. PubMed PMID: 24763065; PMCID: 4059820.
8. Calvo S S, Egan J M. The endocrinology of taste receptors. Nat Rev Endocrinol. 2015; 11(4):213-27. doi: 10.1038/nrendo.2015.7. PubMed PMID: 25707779.
9. Kyriazis G A, Soundarapandian M M, Tyrberg B. Sweet taste receptor signaling in beta cells mediates fructose-induced potentiation of glucose-stimulated insulin secretion. ProcNatlAcadSciUSA. 2012; 109(8): E524-E32. doi: 1115183109 [pii]; 10.1073/pnas.1115183109 [doi].
10. Kyriazis G A, Smith K R, Tyrberg B, Hussain T, Pratley R E. Sweet taste receptors regulate basal insulin secretion and contribute to compensatory insulin hypersecretion during the development of diabetes in male mice. Endocrinology. 2014; 155(6):2112-21. Epub 2014/04/10. doi: 10.1210/en.2013-2015. PubMed PMID: 24712876; PMCID: PMC4020927.
11. Smith K, Karimian Azari E, LaMoia T E, Hussain T, Vargova V, Karolyi K, Veldhuis P P, Arnoletti J P, de la Fuente S G, Pratley R E, Osborne T F, Kyriazis G A. T1R2 receptor-mediated glucose sensing in the upper intestine potentiates glucose absorption through activation of local regulatory pathways. Mol Metab. 2018; 17:98-111. Epub 2018/09/12. doi: 10.1016/j.molmet.2018.08.009. PubMed PMID: 30201274; PMCID: PMC6197762.
12. Smith K R, Hussain T, Karimian Azari E, Steiner J L, Ayala J E, Pratley R E, Kyriazis G A. Disruption of the sugar-sensing receptor T1R2 attenuates metabolic derangements associated with diet-induced obesity. American journal of physiology Endocrinology and metabolism. 2016; 310(8): E688-E98. Epub 2016/02/18. doi: 10.1152/ajpendo.00484.2015. PubMed PMID: 26884387; PMCID: PMC4835941.
13. Stoltzman C A, Peterson C W, Breen K T, Muoio D M, Billin A N, Ayer D E. Glucose sensing by MondoA:Mlx complexes: a role for hexokinases and direct regulation of thioredoxin-interacting protein expression. Proceedings of the National Academy of Sciences of the United States of America. 2008; 105(19):6912-7. Epub 2008/05/07. doi: 10.1073/pnas.0712199105. PubMed PMID: 18458340; PMCID: PMC2383952.
14. Meng Z X, Gong J, Chen Z, Sun J, Xiao Y, Wang L, Li Y, Liu J, Xu X Z S, Lin J D. Glucose Sensing by Skeletal Myocytes Couples Nutrient Signaling to Systemic Homeostasis. Mol Cell. 2017; 66(3):332-44 e4. Epub 2017/05/06. doi: 10.1016/j.molcel.2017.04.007. PubMed PMID: 28475869; PMCID: PMC5489118.
15. Meng Z X, Li S, Wang L, Ko H J, Lee Y, Jung D Y, Okutsu M, Yan Z, Kim J K, Lin J D. Baf60c drives glycolytic metabolism in the muscle and improves systemic glucose homeostasis through Deptor-mediated Akt activation. Nat Med. 2013; 19(5):640-5. Epub 2013/04/09. doi: 10.1038/nm.3144. PubMed PMID: 23563706; PMCID: PMC3650110.
16. Hunt L C, Xu B, Finkelstein D, Fan Y, Carroll P A, Cheng P F, Eisenman R N, Demontis F. The glucose-sensing transcription factor MLX promotes myogenesis via myokine signaling. Genes Dev. 2015; 29(23):2475-89. Epub 2015/11/21. doi: 10.1101/gad.267419.115. PubMed PMID: 26584623; PMCID: PMC4691951.
17. Zhang C S, Hawley S A, Zong Y, Li M, Wang Z, Gray A, Ma T, Cui J, Feng J W, Zhu M, Wu Y Q, Li T Y, Ye Z, Lin S Y, Yin H, Piao H L, Hardie D G, Lin S C. Fructose-1,6-bisphosphate and aldolase mediate glucose sensing by AMPK. Nature. 2017; 548(7665):112-6. Epub 2017/07/21. doi: 10.1038/nature23275. PubMed PMID: 28723898; PMCID: PMC5544942.
18. Canto C, Jiang L Q, Deshmukh A S, Mataki C, Coste A, Lagouge M, Zierath J R, Auwerx J. Interdependence of AMPK and SIRT1 for metabolic adaptation to fasting and exercise in skeletal muscle. Cell Metab. 2010; 11(3):213-9. Epub 2010/03/04. doi: 10.1016/j.cmet.2010.02.006. PubMed PMID: 20197054; PMCID: PMC3616265.
19. Mouchiroud L, Houtkooper R H, Auwerx J. NAD(+) metabolism: a therapeutic target for age-related metabolic disease. Crit Rev Biochem Mol Biol. 2013; 48(4):397-408. Epub 2013/06/08. doi: 10.3109/10409238.2013.789479. PubMed PMID: 23742622; PMCID: PMC3858599.
20. Canto C, Houtkooper R H, Pirinen E, Youn D Y, Oosterveer M H, Cen Y, Fernandez-Marcos P J, Yamamoto H, Andreux P A, Cettour-Rose P, Gademann K, Rinsch C, Schoonjans K, Sauve A A, Auwerx J. The NAD(+) precursor nicotinamide riboside enhances oxidative metabolism and protects against high-fat diet-induced obesity. Cell Metab. 2012; 15(6):838-47. Epub 2012/06/12. doi: 10.1016/j.cmet.2012.04.022. PubMed PMID: 22682224; PMCID: PMC3616313.
21. Costford S R, Brouwers B, Hopf M E, Sparks L M, Dispagna M, Gomes A P, Cornnell H H, Petucci C, Phelan P, Xie H, Yi F, Walter G A, Osborne T F, Sinclair D A, Mynatt R L, Ayala J E, Gardell S J, Smith S R. Skeletal muscle overexpression of nicotinamide phosphoribosyl transferase in mice coupled with voluntary exercise augments exercise endurance. Mol Metab. 2018; 7:1-11. Epub 2017/11/18. doi: 10.1016/j.molmet.2017.10.012. PubMed PMID: 29146412; PMCID: PMC5784330.
22. Brouwers B, Stephens N A, Costford S R, Hopf M E, Ayala J E, Yi F, Xie H, Li J L, Gardell S J, Sparks L M, Smith S R. Elevated Nicotinamide Phosphoribosyl Transferase in Skeletal Muscle Augments Exercise Performance and Mitochondrial Respiratory Capacity Following Exercise Training. Front Physiol. 2018; 9:704. Epub 2018/06/27. doi: 10.3389/fphys.2018.00704. PubMed PMID: 29942262; PMCID: PMC6004371.
23. Fulco M, Cen Y, Zhao P, Hoffman E P, McBurney M W, Sauve A A, Sartorelli V. Glucose restriction inhibits skeletal myoblast differentiation by activating SIRT1 through AMPK-mediated regulation of Nampt. Dev Cell. 2008; 14(5):661-73. Epub 2008/05/15. doi: 10.1016/j.devcel.2008.02.004. PubMed PMID: 18477450; PMCID: PMC2431467.
24. Brandauer J, Vienberg S G, Andersen M A, Ringholm S, Risis S, Larsen P S, Kristensen J M, Frosig C, Leick L, Fentz J, Jorgensen S, Kiens B, Wojtaszewski J F, Richter E A, Zierath J R, Goodyear L J, Pilegaard H, Treebak J T. AMP-activated protein kinase regulates nicotinamide phosphoribosyl transferase expression in skeletal muscle. The Journal of physiology. 2013; 591(20):5207-20. Epub 2013/08/07. doi: 10.1113/jphysiol.2013.259515. PubMed PMID: 23918774; PMCID: PMC3810819.
25. Bai P, Canto C, Brunyanszki A, Huber A, Szanto M, Cen Y, Yamamoto H, Houten S M, Kiss B, Oudart H, Gergely P, Menissier-de Murcia J, Schreiber V, Sauve A A, Auwerx J. PARP-2 regulates SIRT1 expression and whole-body energy expenditure. Cell Metab. 2011; 13(4):450-60. doi: 10.1016/j.cmet.2011.03.013. PubMed PMID: 21459329; PMCID: PMC3108571.
26. Camacho-Pereira J, Tarrago M G, Chini C C S, Nin V, Escande C, Warner G M, Puranik A S, Schoon R A, Reid J M, Galina A, Chini E N. CD38 Dictates Age-Related NAD Decline and Mitochondrial Dysfunction through an SIRT3-Dependent Mechanism. Cell Metab. 2016; 23(6):1127-39. Epub 2016/06/16. doi: 10.1016/j.cmet.2016.05.006. PubMed PMID: 27304511; PMCID: PMC4911708.
27. Escande C, Nin V, Price N L, Capellini V, Gomes A P, Barbosa M T, O'Neil L, White T A, Sinclair D A, Chini E N. Flavonoid apigenin is an inhibitor of the NAD⁺ ase CD38: implications for cellular NAD⁺ metabolism, protein acetylation, and treatment of metabolic syndrome. Diabetes. 2013; 62(4):1084-93. Epub 2012/11/23. doi: 10.2337/dbl2-1139. PubMed PMID: 23172919; PMCID: PMC3609577.
28. Bouche C, Serdy S, Kahn C R, Goldfine A B. The cellular fate of glucose and its relevance in type 2 diabetes. Endocrine reviews. 2004; 25(5):807-30. Epub 2004/10/07. doi: 10.1210/er.2003-0026. PubMed PMID: 15466941.
29. Howarth R E, Baldwin R L. Synthesis and accumulation of protein and nucleic acid in rat gastrocnemius muscles during normal growth, restricted growth, and recovery from restricted growth. The Journal of nutrition. 1971; 101(4):477-84. Epub 1971/04/01. doi: 10.1093/jn/101.4.477. PubMed PMID: 5580068.
30. Grant A L, Skjaerlund D M, Helferich W G, Bergen W G, Merkel R A. Skeletal muscle growth and expression of skeletal muscle alpha-actin mRNA and insulin-like growth factor I mRNA in pigs during feeding and withdrawal of ractopamine. Journal of animal science. 1993; 71(12):3319-26. Epub 1993/12/01. doi: 10.2527/1993.71123319x. PubMed PMID: 8294283.
31. Cabezas H, Raposo R R, Melendez-Hevia E. Activity and metabolic roles of the pentose phosphate cycle in several rat tissues. Mol Cell Biochem. 1999; 201(1-2):57-63. Epub 2000/01/12. doi: 10.1023/a:1007042531454. PubMed PMID: 10630623.
32. Wasserman D H, Kang L, Ayala J E, Fueger P T, Lee-Young R S. The physiological regulation of glucose flux into muscle in vivo. J Exp Biol. 2011; 214(Pt 2):254-62. doi: 10.1242/jeb.048041. PubMed PMID: 21177945; PMCID: PMC3008632.
33. Baron A D, Brechtel G, Wallace P, Edelman S V. Rates and tissue sites of non-insulin- and insulin-mediated glucose uptake in humans. AmJPhysiol. 1988; 255(6 Pt 1): E769-E74.
34. Czech M P. Insulin action and resistance in obesity and type 2 diabetes. Nat Med. 2017; 23(7):804-14. Epub 2017/07/12. doi: 10.1038/nm.4350. PubMed PMID: 28697184; PMCID: PMC6048953.
35. Wu H, Ballantyne C M. Skeletal muscle inflammation and insulin resistance in obesity. The Journal of clinical investigation. 2017; 127(1):43-54. Epub 2017/01/04. doi: 10.1172/JCI88880. PubMed PMID: 28045398; PMCID: PMC5199705 Sanofi-Synthelabo, and has a provisional patent (no. 61/721,475) entitled “Biomarkers to improve prediction of heart failure risk,” filed by Baylor College of Medicine and Roche.
36. Kalyani R R, Corriere M, Ferrucci L. Age-related and disease-related muscle loss: the effect of diabetes, obesity, and other diseases. Lancet Diabetes Endocrinol. 2014; 2(10):819-29. Epub 2014/04/16. doi: 10.1016/S2213-8587(14)70034-8. PubMed PMID: 24731660; PMCID: PMC4156923.
37. Park S W, Goodpaster B H, Lee J S, Kuller L H, Boudreau R, de Rekeneire N, Harris T B, Kritchevsky S, Tylavsky F A, Nevitt M, Cho Y W, Newman A B, Health A, Body Composition S. Excessive loss of skeletal muscle mass in older adults with type 2 diabetes. Diabetes Care. 2009; 32(i 1):1993-7. Epub 2009/06/25. doi: 10.2337/dc09-0264. PubMed PMID: 19549734; PMCID: PMC2768193.
38. Lindstrom P. The physiology of obese-hyperglycemic mice [ob/ob mice]. ScientificWorldJournal. 2007; 7:666-85. Epub 2007/07/11. doi: 10.1100/tsw.2007.117. PubMed PMID: 17619751; PMCID: PMC5901356.
39. Wang B, Chandrasekera P C, Pippin J J. Leptin- and leptin receptor-deficient rodent models: relevance for human type 2 diabetes. Current diabetes reviews. 2014; 10(2):131-45. Epub 2014/05/09. doi: 10.2174/1573399810666140508121012. PubMed PMID: 24809394; PMCID: PMC4082168.
40. Ko H L, Ren E C. Functional Aspects of PARP1 in DNA Repair and Transcription. Biomolecules. 2012; 2(4):524-48. Epub 2012/01/01. doi: 10.3390/biom2040524. PubMed PMID: 24970148; PMCID: PMC4030864.
41. Wise A, Gearing K, Rees S. Target validation of G-protein coupled receptors. Drug discovery today. 2002; 7(4):235-46. PubMed PMID: 11839521.
42. Nakagawa Y, Nagasawa M, Yamada S, Hara A, Mogami H, Nikolaev V O, Lohse M J, Shigemura N, Ninomiya Y, Kojima I. Sweet taste receptor expressed in pancreatic beta-cells activates the calcium and cyclic AMP signaling systems and stimulates insulin secretion. PLoSOne. 2009; 4(4): e5106.
43. Margolskee R F, Dyer J, Kokrashvili Z, Salmon K S, Ilegems E, Daly K, Maillet E L, Ninomiya Y, Mosinger B, Shirazi-Beechey S P. T1R3 and gustducin in gut sense sugars to regulate expression of Na+-glucose cotransporter 1. ProcNatlAcadSciUSA. 2007; 104(38):15075-80.
44. Dyer J, Salmon K S, Zibrik L, Shirazi-Beechey S P. Expression of sweet taste receptors of the T I R family in the intestinal tract and enteroendocrine cells. Biochemical Society transactions. 2005; 33(Pt 1):302-5. doi: 10.1042/BST0330302. PubMed PMID: 15667333.
45. Jang H J, Kokrashvili Z, Theodorakis M J, Carlson O D, Kim B J, Zhou J, Kim H H, Xu X, Chan S L, Juhaszova M, Bernier M, Mosinger B, Margolskee R F, Egan J M. Gut-expressed gustducin and taste receptors regulate secretion of glucagon-like peptide-1. ProcNatlAcadSciUSA. 2007; 104(38):15069-74.
46. Young R L, Chia B, Isaacs N J, Ma J, Khoo J, Wu T, Horowitz M, Rayner C K. Disordered control of intestinal sweet taste receptor expression and glucose absorption in type 2 diabetes. Diabetes. 2013. doi: 10.2337/db13-0581. PubMed PMID: 23761104.
47. Masubuchi Y, Nakagawa Y, Ma J, Sasaki T, Kitamura T, Yamamoto Y, Kurose H, Kojima I, Shibata H. A novel regulatory function of sweet taste-sensing receptor in adipogenic differentiation of 3T3-L1 cells. PloS one. 2013; 8(1).e54500. doi: 10.1371/journal.pone.0054500. PubMed PMID: 23336004; PMCID: 3545961.
48. Simon B R, Parlee S D, Learman B S, Mori H, Scheller E L, Cawthorn W P, Ning X, Gallagher K, Tyrberg B, Assadi-Porter F M, Evans C R, MacDougald OA. Artificial sweeteners stimulate adipogenesis and suppress lipolysis independently of sweet taste receptors. The Journal of biological chemistry. 2013; 288(45):32475-89. doi: 10.1074/jbc.M113.514034. PubMed PMID: 24068707; PMCID: PMC3820882.
49. Nelson G, Hoon M A, Chandrashekar J, Zhang Y, Ryba N J, Zuker C S. Mammalian sweet taste receptors. Cell. 2001; 106(3):381-90.
50. Zhao G Q, Zhang Y, Hoon M A, Chandrashekar J, Erlenbach I, Ryba N J, Zuker C S. The receptors for mammalian sweet and umami taste. Cell. 2003; 115(3):255-66.
51. Ayala J E, Bracy D P, Malabanan C, James F D, Ansari T, Fueger P T, McGuinness O P, Wasserman D H. Hyperinsulinemic-euglycemic clamps in conscious, unrestrained mice. J Vis Exp. 2011(57). doi: 10.3791/3188. PubMed PMID: 22126863; PMCID: PMC3308587.
52. Loffler M G, Birkenfeld A L, Philbrick K M, Belman J P, Habtemichael E N, Booth C J, Castorena C M, Choi C S, Jornayvaz F R, Gassaway B M, Lee H Y, Cartee G D, Philbrick W, Shulman G I, Samuel V T, Bogan J S. Enhanced fasting glucose turnover in mice with disrupted action of TUG protein in skeletal muscle. The Journal of biological chemistry. 2013; 288(28):20135-50. Epub 2013/06/08. doi: 10.1074/jbc.M113.458075. PubMed PMID: 23744065; PMCID: PMC3711282.
53. Tsao T S, Burcelin R, Charron M J. Regulation of hexokinase II gene expression by glucose flux in skeletal muscle. The Journal of biological chemistry. 1996; 271(25):14959-63. Epub 1996/06/21. doi: 10.1074/jbc.271.25.14959. PubMed PMID: 8663067.
54. Sutton J R, Toews C J, Ward G R, Fox I H. Purine metabolism during strenuous muscular exercise in man. Metabolism. 1980; 29(3):254-60. Epub 1980/03/01. doi: 10.1016/0026-0495(80)90067-0. PubMed PMID: 7374440.
55. Stincone A, Prigione A, Cramer T, Wamelink M M, Campbell K, Cheung E, Olin-Sandoval V, Gruning N M, Kruger A, Taugeer Alam M, Keller M A, Breitenbach M, Brindle K M, Rabinowitz J D, Ralser M. The return of metabolism: biochemistry and physiology of the pentose phosphate pathway. Biol Rev Camb Philos Soc. 2015; 90(3):927-63. Epub 2014/09/23. doi: 10.1111/brv.12140. PubMed PMID: 25243985; PMCID: PMC4470864.
56. Liu L, Su X, Quinn W J, 3rd, Hui S, Krukenberg K, Frederick D W, Redpath P, Zhan L, Chellappa K, White E, Migaud M, Mitchison T J, Baur J A, Rabinowitz J D. Quantitative Analysis of NAD Synthesis-Breakdown Fluxes. Cell Metab. 2018; 27(5):1067-80 e5. Epub 2018/04/25. doi: 10.1016/j.cmet.2018.03.018. PubMed PMID: 29685734; PMCID. PMC5932087.
57. Maldonado E N, Lemasters J J. ATP/ADP ratio, the missed connection between mitochondria and the Warburg effect. Mitochondrion. 2014; 19 Pt A:78-84. Epub 2014/09/18. doi: 10.1016/j.mito.2014.09.002. PubMed PMID: 25229666; PMCID: PMC4254332.
58. Fordtran J S, Clodi P H, Soergel K H, Ingelfinger F J. Sugar absorption tests, with special reference to 3-O-methyl-d-glucose and d-xylose. Annals of internal medicine. 1962; 57:883-91. PubMed PMID: 13958713.
59. Karimian Azari E, Smith K R, Yi F, Osborne T F, Bizzotto R, Mari A, Pratley R E, Kyriazis G A. Inhibition of sweet chemosensory receptors alters insulin responses during glucose ingestion in healthy adults: a randomized crossover interventional study. The American journal of clinical nutrition. 2017; 105(4):1001-9. Epub 2017/03/03. doi: 10.3945/ajcn.116.146001. PubMed PMID: 28251932; PMCID: PMC5366051.
60. Sigoillot M, BrockhoffA, Neiers F, Poirier N, Belloir C, Legrand P, Charron C, Roblin P, Meyerhof W, Briand L. The Crystal Structure of Gurmarin, a Sweet Taste-Suppressing Protein: Identification of the Amino Acid Residues Essential for Inhibition. Chemical senses. 2018; 43(8):635-43. doi: 10.1093/chemse/bjy054. PubMed PMID: 30137256.
61. Ninomiya Y, Imoto T. Gurmarin inhibition of sweet taste responses in mice. The American journal of physiology. 1995; 268(4 Pt 2): R1019-25. doi: 10.1152/ajpregu.1995.268.4.R1019. PubMed PMID: 7733384.
62. Zhang Y, Hoon M A, Chandrashekar J, Mueller K L, Cook B, Wu D, Zuker C S, Ryba N J. Coding of sweet, bitter, and umami tastes: different receptor cells sharing similar signaling pathways. Cell. 2003; 112(3):293-301.
63. Luo X, Kraus W L. On PAR with PARP: cellular stress signaling through poly(ADP-ribose) and PARP-1. Genes Dev. 2012; 26(5):417-32. doi: 10.1101/gad.183509.111. PubMed PMID: 22391446; PMCID: PMC3305980.
64. Kauppinen™, Chan W Y, Sub S W, Wiggins A K, Huang E J, Swanson R A. Direct phosphorylation and regulation of poly(ADP-ribose) polymerase-1 by extracellular signal-regulated kinases 1/2. Proceedings of the National Academy of Sciences of the United States of America. 2006; 103(18):7136-41. doi: 10.1073/pnas.0508606103. PubMed PMID: 16627622; PMCID: PMC1459030.
65. Cohen-Armon M, Visochek L, Rozensal D, Kalal A, Geistrikh I, Klein R, Bendetz-Nezer S, Yao Z, Seger R. DNA-independent PARP-1 activation by phosphorylated ERK2 increases Elkl activity: a link to histone acetylation. Mol Cell. 2007; 25(2):297-308. doi: 10.1016/j.molcel.2006.12.012. PubMed PMID: 17244536.
66. Cohen-Armon M. PARP-1 activation in the ERK signaling pathway. Trends Pharmacol Sci. 2007; 28(11):556-60. doi: 10.1016/j.tips.2007.08.005. PubMed PMID: 17950909.
67. Goldsmith Z G, Dhanasekaran D N. G protein regulation of MAPK networks. Oncogene. 2007; 26(22):3122-42. doi: 10.1038/sj.onc.1210407. PubMed PMID: 17496911.
68. Genovese T, Di Paola R, Catalano P, Li J H, Xu W, Massuda E, Caputi A P, Zhang J, Cuzzocrea S. Treatment with a novel poly(ADP-ribose) glycohydrolase inhibitor reduces development of septic shock-like syndrome induced by zymosan in mice. Critical care medicine. 2004; 32(6):1365-74. doi: 10.1097/01.ccm.0000127775.70867.0c. PubMed PMID: 15187521.
69. Frederick D W, Davis J G, Davila A, Jr., Agarwal B, Michan S, Puchowicz M A, Nakamaru-Ogiso E, Baur J A. Increasing NAD synthesis in muscle via nicotinamide phosphoribosyltransferase is not sufficient to promote oxidative metabolism. The Journal of biological chemistry. 2015; 290(3):1546-58. Epub 2014/11/21. doi: 10.1074/jbc.M114.579565. PubMed PMID: 25411251; PMCID: PMC4340401.
70. Hassa P O, Haenni S S, Elser M, Hottiger M O. Nuclear ADP-ribosylation reactions in mammalian cells: where are we today and where are we going? Microbiol Mol Biol Rev. 2006; 70(3):789-829. doi: 10.1128/MMBR.00040-05. PubMed PMID: 16959969; PMCID: PMC1594587.
71. Dong X C. Sirtuin biology and relevance to diabetes treatment. Diabetes Manag (Lond). 2012; 2(3):243-57. doi: 10.2217/dmt.12.16. PubMed PMID: 23024708; PMCID: PMC3458714.
72. Lagouge M, Argmann C, Gerhart-Hines Z, Meziane H, Lerin C, Daussin F, Messadeq N, Milne J, Lambert P, Elliott P, Geny B, Laakso M, Puigserver P, Auwerx J. Resveratrol improves mitochondrial function and protects against metabolic disease by activating SIRT1 and PGC-lalpha. Cell. 2006; 127(6):1109-22. Epub 2006/11/23. doi: 10.1016/j.cell.2006.11.013. PubMed PMID: 17112576.
73. Price N L, Gomes A P, Ling A J, Duarte F V, Martin-Montalvo A, North B J, Agarwal B, Ye L, Ramadori G, Teodoro J S, Hubbard B P, Varela A T, Davis J G, Varamini B, Hafner A, Moaddel R, Rolo A P, Coppari R, Palmeira C M, de Cabo R, Baur J A, Sinclair D A. SIRT1 is required for AMPK activation and the beneficial effects of resveratrol on mitochondrial function. Cell Metab. 2012; 15(5):675-90. Epub 2012/05/09. doi: 10.1016/j.cmet.2012.04.003. PubMed PMID: 22560220; PMCID: PMC3545644.
74. Napper A D, Hixon J, McDonagh T, Keavey K, Pons J F, Barker J, Yau W T, Amouzegh P, Flegg A, Hamelin E, Thomas R J, Kates M, Jones S, Navia M A, Saunders J O, DiStefano P S, Curtis R. Discovery of indoles as potent and selective inhibitors of the deacetylase SIRT1. Journal of medicinal chemistry. 2005; 48(25):8045-54. Epub 2005/12/13. doi: 10.1021/jm050522v. PubMed PMID: 16335928.
75. Peck B, Chen C Y, Ho K K, Di Fruscia P, Myatt S S, Coombes R C, Fuchter M J, Hsiao C D, Lam E W. SIRT inhibitors induce cell death and p53 acetylation through targeting both SIRT1 and SIRT2. Mol Cancer Ther. 2010; 9(4):844-55. Epub 2010/04/08. doi: 10.1158/1535-7163.MCT-09-0971. PubMed PMID: 20371709.
76. Sharabi K, Lin H, Tavares C D J, Dominy J E, Camporez J P, Perry R J, Schilling R, Rines A K, Lee J, Hickey M, Bennion M, Palmer M, Nag P P, Bittker J A, Perez J, Jedrychowski M P, Ozcan U, Gygi S P, Kamenecka™, Shulman G I, Schreiber S L, Griffin P R, Puigserver P. Selective Chemical Inhibition of PGC-lalpha Gluconeogenic Activity Ameliorates Type 2 Diabetes. Cell. 2017; 169(1):148-60 e15. Epub 2017/03/25. doi: 10.1016/j.cell.2017.03.001. PubMed PMID: 28340340; PMCID: PMC5398763.
77. Gerhart-Hines Z, Rodgers J T, Bare O, Lerin C, Kim S H, Mostoslavsky R, Alt F W, Wu Z, Puigserver P. Metabolic control of muscle mitochondrial function and fatty acid oxidation through SIRT1/PGC-lalpha. EMBO J. 2007; 26(7):1913-23. doi: 10.1038/sj.emboj.7601633. PubMed PMID: 17347648; PMCID: PMC1847661.
78. Rodgers J T, Lerin C, Haas W, Gygi S P, Spiegelman B M, Puigserver P. Nutrient control of glucose homeostasis through a complex of PGC-lalpha and SIRT1. Nature. 2005; 434(7029):113-8. doi: 10.1038/nature03354. PubMed PMID: 15744310.
79. Pesta D, Gnaiger E. High-resolution respirometry: OXPHOS protocols for human cells and permeabilized fibers from small biopsies of human muscle. Methods Mol Biol. 2012; 810:25-58. Epub 2011/11/08. doi: 10.1007/978-1-61779-382-O_3. PubMed PMID: 22057559.
80. Lantier L, Williams A S, Hughey C C, Bracy D P, James F D, Ansari M A, Gius D, Wasserman D H. SIRT2 knockout exacerbates insulin resistance in high fat-fed mice. PloS one. 2018; 13(12): e0208634. Epub 2018/12/12. doi: 10.1371/journal.pone.0208634. PubMed PMID: 30533032; PMCID: PMC6289500.
81. Belman J P, Bian R R, Habtemichael E N, Li D T, Jurczak M J, Alcazar-Roman A, McNally L J, Shulman G I, Bogan J S. Acetylation of TUG protein promotes the accumulation of GLUT4 glucose transporters in an insulin-responsive intracellular compartment. The Journal of biological chemistry. 2015; 290(7):4447-63. Epub 2015/01/07. doi: 10.1074/jbc.M114.603977. PubMed PMID: 25561724; PMCID: PMC4326849.
82. Wang Y P, Zhou L S, Zhao Y Z, Wang S W, Chen L L, Liu L X, Ling Z Q, Hu F J, Sun Y P, Zhang J Y, Yang C, Yang Y, Xiong Y, Guan K L, Ye D. Regulation of G6PD acetylation by SIRT2 and KAT9 modulates NADPH homeostasis and cell survival during oxidative stress. EMBO J. 2014; 33(12):1304-20. Epub 2014/04/29. doi: 10.1002/embj.201387224. PubMed PMID: 24769394; PMCID: PMC4194121.
83. Wu L E, Sinclair D A. SIRT2 controls the pentose phosphate switch. EMBO J. 2014; 33(12):1287-8. Epub 2014/05/16. doi: 10.15252/embj.201488713. PubMed PMID: 24825350; PMCID: PMC4194117.
84. Xu S N, Wang T S, Li X, Wang Y P. SIRT2 activates G6PD to enhance NADPH production and promote leukaemia cell proliferation. Sci Rep. 2016; 6:32734. Epub 2016/09/03. doi: 10.1038/srep32734. PubMed PMID: 27586085; PMCID: PMC5009355.
85. Lane A N, Fan T W. Regulation of mammalian nucleotide metabolism and biosynthesis. Nucleic acids research. 2015; 43(4):2466-85. doi: 10.1093/nar/gkv047. PubMed PMID: 25628363; PMCID: PMC4344498.
86. Spiegelman N A, Price I R, Jing H, Wang M, Yang M, Cao J, Hong J Y, Zhang X, Aramsangtienchai P, Sadhukhan S, Lin H. Direct Comparison of SIRT2 Inhibitors: Potency, Specificity, Activity-Dependent Inhibition, and On-Target Anticancer Activities. ChemMedChem. 2018; 13(18):1890-4. Epub 2018/07/31. doi: 10.1002/cmdc.201800391. PubMed PMID: 30058233; PMCID: PMC6402572.
87. Hinkley J M, Ferey J L, Brault J J, Smith C A, Gilliam L A, Witczak C A. Constitutively active CaMKKalpha stimulates skeletal muscle glucose uptake in insulin-resistant mice in vivo. Diabetes. 2014; 63(1):142-51. Epub 2013/10/09. doi: 10.2337/db13-0452. PubMed PMID: 24101676; PMCID: PMC3868053.
88. Wensaas A J, Rustan A C, Lovstedt K, Kull B, Wikstrom S, Drevon C A, Hallen S. Cell-based multiwell assays for the detection of substrate accumulation and oxidation. Journal of lipid research. 2007; 48(4):961-7. Epub 2007/01/11. doi: 10.1194/jlr.D600047-JLR200. PubMed PMID: 17213484.
89. Lin S C, Hardie D G. AMPK: Sensing Glucose as well as Cellular Energy Status. Cell Metab. 2018; 27(2):299-313. doi: 10.1016/j.cmet.2017.10.009. PubMed PMID: 29153408.
90. Dibble C C, Manning B D. Signal integration by mTORC1 coordinates nutrient input with biosynthetic output. Nat Cell Biol. 2013; 15(6):555-64. doi: 10.1038/ncb2763. PubMed PMID: 23728461; PMCID: PMC3743096.
91. Wauson E M, Zaganjor E, Lee A Y, Guerra M L, Ghosh A B, Bookout A L, Chambers C P, Jivan A, McGlynn K, Hutchison M R, Deberardinis R J, Cobb M H. The G protein-coupled taste receptor T1 R1/T1R3 regulates mTORC1 and autophagy. Mol Cell. 2012; 47(6):851-62. doi: 10.1016/j.molcel.2012.08.001. PubMed PMID: 22959271; PMCID: PMC3749915.
92. Ju B G, Solum D, Song E J, Lee K J, Rose D W, Glass C K, Rosenfeld M G. Activating the PARP-1 sensor component of the groucho/TLE1 corepressor complex mediates a CaMKinase IIdelta-dependent neurogenic gene activation pathway. Cell. 2004; 119(6):815-29. doi: 10.1016/j.cell.2004.11.017. PubMed PMID: 15607978.
93. Pardo P S, Boriek A M. The physiological roles of Sirt1 in skeletal muscle. Aging (Albany NY). 2011; 3(4):430-7. Epub 2011/04/13. doi: 10.18632/aging.100312. PubMed PMID: 21483036; PMCID: PMC3117458.
94. Gurd B J, Holloway G P, Yoshida Y, Bonen A. In mammalian muscle, SLRT3 is present in mitochondria and not in the nucleus; and SIRT3 is upregulated by chronic muscle contraction in an adenosine monophosphate-activated protein kinase-independent manner. Metabolism. 2012; 61(5):733-41. doi: 10.1016/j.metabol.2011.09.016. PubMed PMID: 22078938.
95. Hokari F, Kawasaki E, Sakai A, Koshinaka K, Sakuma K, Kawanaka K. Muscle contractile activity regulates Sirt3 protein expression in rat skeletal muscles. J Appl Physiol (1985). 2010; 109(2):332-40. doi: 10.1152fjapplphysiol.00335.2009. PubMed PMID: 20413424.
96. Palacios O M, Carmona J J, Michan S, Chen K Y, Manabe Y, Ward J L, 3rd, Goodyear L J, Tong Q. Diet and exercise signals regulate SIRT3 and activate AMPK and PGC-lalpha in skeletal muscle. Aging (Albany NY). 2009; 1(9):771-83. doi: 10.18632/aging.100075. PubMed PMID: 20157566; PMCID: PMC2815736.
97. Jing E, O'Neill B T, Rardin M J, Kleinridders A, Ilkeyeva O R, Ussar S, Bain J R, Lee K Y, Verdin E M, Newgard C B, Gibson B W, Kahn C R. Sirt3 regulates metabolic flexibility of skeletal muscle through reversible enzymatic deacetylation. Diabetes. 2013; 62(10):3404-17. Epub 2013/07/10. doi: 10.2337/dbl2-1650. PubMed PMID: 23835326; PMCID: PMC3781465.
98. Lantier L, Williams A S, Williams I M, Yang K K, Bracy D P, Goelzer M, James F D, Gius D, Wasserman D H. SIRT3 Is Crucial for Maintaining Skeletal Muscle Insulin Action and Protects Against Severe Insulin Resistance in High-Fat-Fed Mice. Diabetes. 2015; 64(9):3081-92. Epub 2015/05/08. doi: 10.2337/dbl4-1810. PubMed PMID: 25948682; PMCID: PMC4542443.
99. Jing E, Emanuelli B, Hirschey M D, Boucher J, Lee K Y, Lombard D, Verdin E M, Kahn C R. Sirtuin-3 (Sirt3) regulates skeletal muscle metabolism and insulin signaling via altered mitochondrial oxidation and reactive oxygen species production. Proceedings of the National Academy of Sciences of the United States of America. 2011; 108(35):14608-13. Epub 2011/08/30. doi: 10.1073/pnas.1111308108. PubMed PMID: 21873205; PMCID: PMC3167496.
100. Dolle C, Rack J G, Ziegler M. NAD and ADP-ribose metabolism in mitochondria. FEBS J. 2013; 280(15):3530-41. Epub 2013/04/27. doi: 10.1111/febs.12304. PubMed PMID: 23617329.
101. Goodman C A, Mabrey D M, Frey J W, Miu M H, Schmidt E K, Pierre P, Hornberger T A. Novel insights into the regulation of skeletal muscle protein synthesis as revealed by a new nonradioactive in vivo technique. FASEB J. 2011; 25(3):1028-39. doi: 10.1096/fj.10-168799. PubMed PMID: 21148113; PMCID: PMC3042844.
102. Jackson K C, Tarpey M D, Valencia A P, Inigo M R, Pratt S J, Patteson D J, McClung J M, Lovering R M, Thomson D M, Spangenburg E E. Induced Cre-mediated knockdown of Brcal in skeletal muscle reduces mitochondrial respiration and prevents glucose intolerance in adult mice on a high-fat diet. FASEB J. 2018; 32(6):3070-84. Epub 2018/02/07. doi: 10.1096/fj.201700464R. PubMed PMID: 29401626; PMCID: PMC5956240.
103. Lecker S H, Solomon V, Mitch W E, Goldberg A L. Muscle protein breakdown and the critical role of the ubiquitin-proteasome pathway in normal and disease states. The Journal of nutrition. 1999; 129(1S Suppl):227S-37S. Epub 1999/01/23. doi: 10.1093/jn/129.1.227S. PubMed PMID: 9915905.
104. Goodman C A, Kotecki J A, Jacobs B L, Hornberger T A. Muscle fiber type-dependent differences in the regulation of protein synthesis. PoS one. 2012; 7(5): e37890. Epub 2012/05/26. doi: 10.1371/journal.pone.0037890. PubMed PMID: 22629468; PMCID: PMC3358270.
105. Hoppeler H. Molecular networks in skeletal muscle plasticity. J Exp Biol. 2016; 219(Pt 2):205-13. doi: 10.1242/jeb.128207. PubMed PMID: 26792332.
106. Schiaffino S, Dyar K A, Ciciliot S, Blaauw B, Sandri M. Mechanisms regulating skeletal muscle growth and atrophy. FEBS J. 2013; 280(17):4294-314. Epub 2013/03/23. doi: 10.1111/febs.12253. PubMed PMID: 23517348.
107. Terena S M, Fernandes K P, Bussadori S K, Deana A M, Mesquita-Ferrari R A. Systematic review of the synergist muscle ablation model for compensatory hypertrophy. Rev Assoc Med Bras (1992). 2017; 63(2):164-72. Epub 2017/03/30. doi: 10.1590/1806-9282.63.02.164. PubMed PMID: 28355378.
108. Gao Y, Arfat Y, Wang H, Goswami N. Muscle Atrophy Induced by Mechanical Unloading: Mechanisms and Potential Countermeasures. Front Physiol. 2018; 9:235. Epub 2018/04/05. doi: 10.3389/fphys.2018.00235. PubMed PMID: 29615929; PMCID: PMC5869217.
109. Lang S M, Kazi A A, Hong-Brown L, Lang C H. Delayed recovery of skeletal muscle mass following hindlimb immobilization in mTOR heterozygous mice. PloS one. 2012; 7(6): e38910. Epub 2012/06/30. doi: 10.1371journal.pone.0038910. PubMed PMID: 22745686; PMCID: PMC3382153.
110. Kirby T J, McCarthy J J, Peterson C A, Fry C S. Synergist Ablation as a Rodent Model to Study Satellite Cell Dynamics in Adult Skeletal Muscle. Methods Mol Biol. 2016; 1460:43-52. Epub 2016/08/06. doi: 10.1007/978-1-4939-3810-0_4. PubMed PMID: 27492164.
111. Goodman C A, Pierre P, Hornberger T A. Imaging of protein synthesis with puromycin. Proceedings of the National Academy of Sciences of the United States of America. 2012; 109(17): E989; author reply E90. Epub 2012/03/27. doi: 10.1073/pnas.1202000109. PubMed PMID: 22447778; PMCID: PMC3340059.
112. Bodine S C, Stitt T N, Gonzalez M, Kline W O, Stover G L, Bauerlein R, Zlotchenko E, Scrimgeour A, Lawrence J C, Glass D J, Yancopoulos G D. Akt/mTOR pathway is a crucial regulator of skeletal muscle hypertrophy and can prevent muscle atrophy in vivo. Nat Cell Biol. 2001; 3(11):1014-9. Epub 2001/11/21. doi: 10.1038/ncb1101-1014. PubMed PMID: 11715023.
113. Sanz E, Yang L, Su T, Morris D R, McKnight G S, Amieux P S. Cell-type-specific isolation of ribosome-associated mRNA from complex tissues. Proceedings of the National Academy of Sciences of the United States of America. 2009; 106(33):13939-44. Epub 2009/08/12. doi: 10.1073/pnas.0907143106. PubMed PMID: 19666516; PMCID: PMC2728999.
114. Anderson J E, Wozniak A C, Mizunoya W. Single muscle-fiber isolation and culture for cellular, molecular, pharmacological, and evolutionary studies. Methods Mol Biol. 2012; 798:85-102. Epub 2011/12/02. doi: 10.1007/978-1-61779-343-1_6. PubMed PMID: 22130833.
115. Krause M P, Moradi J, Coleman S K, D'Souza D M, Liu C, Kronenberg M S, Rowe D W, Hawke T J, Hadjiargyrou M. A novel GFP reporter mouse reveals Mustnl expression in adult regenerating skeletal muscle, activated satellite cells and differentiating myoblasts. Acta Physiol (Oxf). 2013; 208(2):180-90. Epub 2013/03/20. doi: 10.1111/apha.12099. PubMed PMID: 23506283.
116. Ontell M, Feng K C, Klueber K, Dunn R F, Taylor F. Myosatellite cells, growth, and regeneration in murine dystrophic muscle: a quantitative study. Anat Rec. 1984; 208(2):159-74. Epub 1984/02/01. doi: 10.1002/ar.1092080203. PubMed PMID: 6703334.
117. Rosenblatt J D, Parry D J. Gamma irradiation prevents compensatory hypertrophy of overloaded mouse extensor digitorum longus muscle. J AppI Physiol (1985). 1992; 73(6):2538-43. Epub 1992/12/01. doi: 10.1152/jappl.1992.73.6.2538. PubMed PMID: 1490967.
118. Agbulut O, Noirez P, Beaumont F, Butler-Browne G. Myosin heavy chain isoforms in postnatal muscle development of mice. Biol Cell. 2003; 95(6):399-406. Epub 2003/10/02. doi: 10.1016/s0248-4900(03)00087-x. PubMed PMID: 14519557.
119. Hirata Y, Nomura K, Senga Y, Okada Y, Kobayashi K, Okamoto S, Minokoshi Y, Imamura M, Takeda S, Hosooka T, Ogawa W. Hyperglycemia induces skeletal muscle atrophy via a WWPl/KLF15 axis. JCI Insight. 2019; 4(4). Epub 2019/03/05. doi: 10.1172/jci.insight. 124952. PubMed PMID: 30830866; PMCID: PMC6478420.
120. Goldspink G. Muscle growth and muscle function: a molecular biological perspective. Res Vet Sci. 1996; 60(3):193-204. doi: 10.1016/s0034-5288(96)90038-7. PubMed PMID: 8735506.
121. Goldspink G, Scutt A, Martindale J, Jaenicke T, Turay L, Gerlach G F. Stretch and force generation induce rapid hypertrophy and myosin isoform gene switching in adult skeletal muscle. Biochemical Society transactions. 1991; 19(2):368-73. doi: 10.1042/bst0190368. PubMed PMID: 1716229.
122. Lowe D A, Alway S E. Animal models for inducing muscle hypertrophy: are they relevant for clinical applications in humans? J Orthop Sports Phys Ther. 2002; 32(2):36-43. doi: 10.2519/jospt.2002.32.2.36. PubMed PMID: 11838579.

1. A method of modulating cell metabolism in skeletal muscle in a subject in need thereof, the method comprising:
a. Identifying a subject in need of modulation of cell metabolism in skeletal muscle; and
b. administering to the subject a modulator of skeletal muscle in T1R2.

2. The method of claim 1, wherein modulation of the T1R2 comprises inhibition or downregulation of T1R2.

3. The method of claim 2, wherein said inhibition or downregulation comprises abrogation of glucose sensing by T1R2.

4. The method of claim 3, wherein said modulator downregulates the STR or a component thereof by 50% or more compared to non-modulated T1R2.

5. The method of claim 1, wherein said modulation of cell metabolism comprises modulation of intracellular NAD levels, Poly ADP ribose polymerases, PARP1 activity, mitochondrial function, oxidative capacity, exercise tolerance, or muscle fiber mass and/or size.

6-11. (canceled)

12. The method of claim 1, wherein said T1R2 is specific to skeletal muscle.

13. The method of claim 1, wherein said T1R2 is found in skeletal muscle as well as other locations in the subject.

14. The method of claim 1, wherein said modulator is a small molecule.

15. The method of claim 14, wherein said small molecules are antagonists or inverse agonist derived from or structurally related to sucrose, glucose, sucralose, saccharin, aspartame, neotame, brazzein, miraculin, S-819, perillartine, P-4000, SE-1, SE-2 (FEMA 4669), SE-3, SE-4, amiloride or gurmarin.

16. The method of claim 14, wherein said small molecule is an antagonist or inverse agonist derived from or structurally related to tas1r2 ligands

17. The method of claim 1, wherein said modulator is a nucleic acid inhibitor.

18. The method of claim 17, wherein said nucleic acid inhibitor is small interfering RNA (siRNA).

19. The method of claim 18, wherein the nucleic acid inhibitor is sc-40196.

20. The method of claim 18, wherein the nucleic acid inhibitor is ORIGENE shRNA CAT #: TL505429V.

21. The method of claim 17, wherein said nucleic acid inhibition is accomplished through CRISPR technology.

22. The method of claim 1, wherein the subject has been diagnosed with muscle wasting or has been diagnosed with being at risk of muscle wasting.

23. The method of claim 22, wherein the muscle wasting is caused by cancer, obesity, metabolic dysfunction, aging, age-related sarcopenia, and/or disuse atrophy.

24. The method of claim 1, wherein the modulator does not affect taste in the subject.

25. The method of claim 1, wherein the modulator is given via an injection.

26. The method of claim 25, wherein the injection is intramuscular.

27. The method of claim 1, wherein the modulator is given via an intravenous drip.

28. The method of claim 1, wherein the modulator is not given to the subject orally.

29. The method of claim 1, wherein the modulator is given to the subject orally.

30-39. (canceled)
